(19) United States
(12) Patent Application Publication
(10) Pub. No.: US 2026/0120890 A1
(43) Pub. Date: Apr. 30, 2026

(54) EFFICIENT QUERYING WITH DIVERSELY ENCODED CLINICAL DATA

(71) Applicant: Oracle International Corporation, Redwood Shores, CA (US)

(72) Inventor: Raman Grover, Palo Alto, CA (US)

(73) Assignee: Oracle International Corporation, Redwood Shores, CA (US)

(21) Appl. No.: 19/368,448

(22) Filed: Oct. 24, 2025

Related U.S. Application Data
(60) Provisional application No. 63/711,943, filed on Oct. 25, 2024.

(51) Int. Cl. G16H 50/70; G06F 16/22; G06F 16/242; G06F 16/2457; G06F 16/901

(52) U.S. Cl. CPC G16H 50/70; G06F 16/2255; G06F 16/2428; G06F 16/24578; G06F 16/9024

(57) ABSTRACT

Techniques are disclosed for querying with semantic code expansion in a clinical data system. In one aspect, a method includes receiving a query containing a predicate specifying a clinical code and a semantic expansion parameter indicating a request for approximate matching. A vector embedding associated with the specified clinical code is retrieved from a pre-computed embedding index. A similarity search is performed in a vector space to identify semantically similar codes. Exact code mappings are retrieved for the specified clinical code from a mapping registry. A rewritten query predicate is generated include the exact code mappings and the semantically similar clinical code mappings. The rewritten query is executed against a clinical data store to retrieve results matching the exact and/or semantically similar codes. The results are annotated to distinguish between exact and semantic matches.

### CROSS-REFERENCE TO RELATED APPLICATION

The present application is a non-provisional application of and claims the benefit and priority under 55 U.S.C. 119 (e) of U.S. Application 63/711,943, filed on Oct. 25, 2024, the disclosure of which is incorporated herein by reference in its entirety for all purposes.

### FIELD

The present disclosure relates generally to data systems, and more particularly, to techniques for improved data processing and querying in clinical data systems.

### BACKGROUND

Heterogeneous and disparate data stores can make computing and querying data more flexible and efficient. Applications that interface with data storage systems can better query data that suit their needs, rather than being limited to a particular type of data query or store. The data storage system can also scale better to better optimize for different workloads.

In recent years, there has been a significant rise in capabilities of data storage systems. In particular, improvements in natural language processing (NLP) have increased the abilities of data storage systems to store semantic concepts of data stored with the storage system. Managing and processing data across various components, particularly for data storage systems with disparate data stores, data models, or the like can be difficult to maintain.

The improvement of data storage systems represents a significant advancement in making data storage systems more accessible and accurate. By improving capabilities of data storage systems, these systems can improve access to information across applications. This disclosure presents techniques related to improved data processing techniques in clinical data systems.

### BRIEF SUMMARY

Data processing techniques are disclosed herein (e.g., computer-implemented methods, systems, non-transitory computer-readable media storing code or instructions executable by one or more processors) for efficient querying of diversly encoded clinical data stored in a clinical data system.

In some embodiments, a computer-implemented for execution queries with semantic code expansion in a clinical data system includes receiving a query containing a predicate specifying a clinical code and a semantic expansion parameter indicating a request for approximate matching; retrieving a vector embedding associated with the specified clinical code from a pre-computed embedding index; performing, based on the vector embedding, a similarity search in a vector space to identify one or more semantically similar clinical codes; retrieving one or more exact code mappings for the specified clinical code from a mapping registry; generating a rewritten query predicate comprising (i) the one or more exact code mappings as primary match conditions and (ii) the one or more semantically similar clinical codes as secondary match conditions; executing the rewritten query against a clinical data store to return results matching (i) the one or more exact code mappings, (ii) the one or more semantically similar clinical codes, or (iii) both; and annotating the results to distinguish between exact matches and semantic matches.

In some embodiments, performing the similarity search comprises: computing a set of similarity scores wherein each similarity score of the set of similarity scores is computed between the retrieved vector embedding and a vector embedding of a candidate code; and selecting the one or more semantically similar clinical codes by identifying one or more similarity scores of the set of similarity scores that exceed a threshold.

In some embodiments, the mapping registry comprises a mapping table including hash-based indexes, and wherein retrieving the one or more exact code mappings comprises performing a point lookup based on a hash index of the clinical code.

In some embodiments, the mapping registry comprises a hierarchical graph stored in a graph database, and wherein retrieving the one or more exact code mappings comprises performing a traversal of the hierarchical graph based on the clinical code and one or more coding systems to identify the one or more exact code mappings.

In some embodiments, the query comprises one or more constructs indicating at least one of (i) a coding system associated with the clinical code or (ii) a target coding system for approximate matching.

In some embodiments, the semantic expansion parameter indicates at least one of (i) a type of semantic expansion or (ii) a number of candidate codes.

In some embodiments, the query indicates a specified version of the clinical code, wherein the specified version corresponds to a historical value of the clinical code; retrieving the one or more exact code mappings comprises retrieving one or more exact code mappings associated with the specified version of the clinical code; the retrieved vector embedding is associated with the specified version of the clinical code; and the results are annotated with mapping version metadata.

In some embodiments, the computer-implemented method further includes:
determining, for each result matching the one or more semantically similar codes, a relevance score; and annotating the results with the relevance score for each semantic match.

In some embodiments, the clinical data system comprises a plurality of clinical data stores, and the computer-implemented method further includes: retrieving results matching the exact code mappings from a relational data store of the plurality of clinical data stores; and retrieving results matching the semantically similar clinical codes from a vector data store of the plurality of clinical data stores.

Some embodiments include a system that includes one or more processors; and one or more computer-readable media storing instructions which, when executed by the one or more processors, cause the system to perform part or all of the operations and/or methods disclosed herein.

Some embodiments include one or more non-transitory computer-readable media storing instructions which, when executed by one or more processors, cause a system to perform part or all of the operations and/or methods disclosed herein.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

FIG. 1 is an example of an architecture for a computing environment for a clinical digital assistant in accordance with various embodiments.

FIG. 2 is a block diagram of a digital assistant runtime flow with components and

interfaces into a semantic index, in accordance with various embodiments.

FIG. 3 is a simplified block diagram of a computing environment of a disparate data storage system, in accordance with various embodiments.

FIG. 4 is an example of an architecture for a computing environment for a data storage system implemented with an ingestion flow for disparate data stores, in accordance with various embodiments.

FIG. 5 is a simplified block diagram of a watermark generation and evaluation within a data ingestion flow, in accordance with various embodiments.

FIG. 6 is a block diagram illustrating an exemplary computing environment implementing semantic translation and expansion of clinical codes, in accordance with various embodiments.

FIG. 7 depicts an example flow for a dynamic direct clinical translation by a clinical data system, in accordance with various embodiments.

FIG. 8 depicts an example flow for processing a query with dynamic translation and semantic expansion, in accordance with various embodiments.

FIG. 9 is a flowchart of a process for executing queries with semantic code expansion in a clinical data system, in accordance with various embodiments.

FIG. 10 is a block diagram illustrating one pattern for implementing a cloud infrastructure as a service system, in accordance with various embodiments.

FIG. 11 is a block diagram illustrating another pattern for implementing a cloud infrastructure as a service system, in accordance with various embodiments.

FIG. 12 is a block diagram illustrating another pattern for implementing a cloud infrastructure as a service system, in accordance with various embodiments.

FIG. 13 is a block diagram illustrating another pattern for implementing a cloud infrastructure as a service system, in accordance with various embodiments.

FIG. 14 is a block diagram illustrating an example computer system, in accordance with various embodiments.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

### INTRODUCTION

In recent years, the amount of data powering various industries and their systems has been increasing exponentially. Organizations and businesses store and consume data across various types of data stores (e.g., relational databases, non-relational databases, object stores, key-value stores, file storage, etc.). These data stores power information systems across multiple industries, for instance, consumer tech (e.g., orders, cancellations, refunds), supply chain (e.g., raw materials, stocks, vendors), healthcare (e.g., medical records), finance (e.g., financial business metrics), customer support, search engines, and much more. Data that powers these industries can come from a variety of different sources and it is imperative for modern data-driven organizations to maintain consistent and reliable data to provide accurate representations of data to users.

With the rise of natural language (NL) processing and artificial intelligence capabilities, storing and providing data in ways that maintain semantic coherence and meaning can improve user queries and interactions with data storage systems. It is vastly more efficient for non-technical users (e.g., business leaders, doctors, or other users of the data) directly interact with analytics tables via natural language (NL) queries that abstract away underlying query language and/or data structures of a data storage system. Further, for data storage systems with multiple sources of data reflected within the storage systems, querying the system with a single unified structure can make accessing data more efficient and reduce user burden. By providing unified query and storage structures, even technical users with strong understandings of one type of data storage but lacking knowledge in other types of data storage can better query a data storage system based on types of data storage and querying implementations they are comfortable with.

Implementing a Semantic Object Model in a data environment with disparate and/or distributed data stores can be a powerful tool for unifying data across disparate and/or distributed data stores while providing efficient access to data. Unlike other data models, which often only define objects by structure, a Semantic Object Model can define objects by their semantic meaning and relationships. Objects are represented as concepts associated with various attributes and relationships that can be leveraged to determine semantics and meaning. For example, in a healthcare environment, a patient can be represented as a concept, and semantic objects corresponding to a patient concept can include various attributes that can describe the patient such as name, address, phone number, and the like. In some implementations, semantic objects can include self-describing metadata and/or linked actions for custom operations.

In healthcare and clinical environments, a heterogeneous data system implementing a Semantic Object Model can be incredibly beneficial for providing healthcare providers unified and comprehensive access to clinical data. Because clinical data can take many shapes such as clinical notes, diagnosis information, labs, medication, etc., a heterogeneous clinical data system with various types of data stores can significantly improve storage and access to data. In particular, leveraging a Semantic Object Model within such heterogeneous data systems can enable applications and frameworks that interact with the clinical data system to provide better abstraction to users and reduce complexities in understanding data by enabling semantic analysis and reasoning.

A particular challenge in accessing clinical data, especially in clinical data systems that include and ingest data from a multitude of sources, however, is using and understanding various clinical codes used to represent medical concepts. Clinical data systems often encode medical concepts (e.g., diagnoses, procedures, medications, etc.) using diverse standardized coding systems. Examples of clinical coding systems used by various clinical data systems include Systemized Nomenclature of Medicine—Clinical Terms (SNOMED CT), International Classification of Diseases (ICD-10), Unified Medical Language System (UMLS), and Logical Observation Identifiers Names and Codes (LOINC). The use of these clinical codes can help maintain consistent information and standardized records in electronic health records, enabling more dependable record keeping and data analysis. While each individual clinical coding system provides a standard method of identifying medical concepts without referring to them with inconsistent free-text description, each clinical coding system can represent medical concepts in vastly different ways. Beyond representing identical clinical concepts with different clinical codes, clinical concepts in different clinical coding systems are often linked with varying numbers of clinical codes based on the organizational or hierarchical structure of the clinical coding system. Some clinical coding systems assign clinical codes to fairly broad medical concepts, while other clinical coding systems assign clinical codes very granular and particular medical concepts.

Requests and analysis of clinical data are often performed using a clinical code representing the relevant medical concept. When querying data from a clinical data system or exchanging data across clinical data systems, differences in codes across coding systems can make it challenging for users to properly understand and interact with the clinical data. For the above example for Type 2 diabetes, a user or entity (e.g., application, etc.) that typically interacts with clinical data encoded with SNOMED clinical codes may have difficulty in understanding both the actual clinical code values of an ICD-10 clinical coding system and the manner in which medical concepts are organized. To resolve these differences in clinical codes across clinical coding systems, applications that interact with clinical data complying with various clinical coding systems typically use explicit application-level code to handle translations between clinical coding systems. This can include managing static lookup tables that map clinical codes across the clinical coding systems. However, this management of clinical code by application-level programs can be prone to errors and increase risks of inconsistencies when using clinical data and reduces the interoperability of a clinical data system across different applications. Some applications even use external services for translations, which can complicate data transformation pipelines for ingesting and interacting with clinical data.

Furthermore, clinical codes and associated definitions for coding systems are updated on a fairly consistent basis (e.g., annually for many clinical coding systems). These updates can increase challenges in user understanding of new clinical codes and in mapping concepts between different versions of clinical codes. As such, reproducing past queries based on previous clinical code versions can be important to ensure clinical decisions can be retraced to know what information was used to make certain decisions. Changes in definitions of clinical codes can cause differences in clinical data retrieve using the clinical code, which can be detrimental in healthcare decision making.

Moreover, when retrieving clinical data from a clinical data system, it can be helpful to understand concepts related to the clinical code beyond retrieving information directly about the code. In cases of translation of codes across coding systems, direct mappings may not entirely equip a user with requested clinical information. For example, a certain disease may be represented by a particular clinical code in a source coding system, but may be represent using a different granularity and/or hierarchy in a target coding system. To fully understand information about the disease, retrieving clinical data only related to the exact mapping of the clinical code may neglect clinical data that is not determined to map exactly to the source clinical code, but may still be associated with semantically similar information.

To address these challenges and others, a technical solution involving dynamic query translation and semantic expansion of clinical codes has been developed. Optionally, the query can include a construct that specifies a source clinical coding system of the clinical code and the source clinical coding system may be different and/or the same as the clinical coding system implemented by the clinical data system. The query can additionally include a semantic expansion parameter the indicates a request for approximate matching of the specified clinical code and clinical codes of the clinical data system. Retrieval of clinical data within the clinical data system using exact code mappings and approximate matches can be integrated by rewriting a query to include both exact code mappings and approximate code matches. This can enable retrieving results that are both exact and semantically similar with a single query. A query result can be annotated to include an indication of a particular result being an exact match or an approximate match.

In one exemplary embodiment, a computer-implemented method for executing queries with semantic code expansion in a clinical data system is provided that includes receiving a query containing a predicate specifying a clinical code and a semantic expansion parameter indicating a request for approximate matching; retrieving a vector embedding associated with the specified clinical code from a pre-computed embedding index; performing, based on the vector embedding, a similarity search in a vector space to identify one or more semantically similar clinical codes; retrieving one or more exact code mappings for the specified clinical code from a mapping registry; generating a rewritten query predicate comprising (i) the one or more exact code mappings as primary match conditions and (ii) the one or more semantically similar clinical codes as secondary match conditions; executing the rewritten query against a clinical data store to return results matching (i) the one or more exact code mappings, (ii) the one or more semantically similar clinical codes, or (iii) both; and annotating the results to distinguish between exact matches and semantic matches.

The implementation of query rewriting within the clinical data system for translating clinical codes across coding systems directly addresses the challenges with application-level mapping logic and enables interoperability across applications by enabling processing of queries using various clinical coding systems without application-level modification. Furthermore, the integration of approximate and exact matches through semantic code expansion enables users to interact with direct code mappings and semantic mappings, which provides technical improvements unified clinical data retrieval. By providing unified types of matches, users can receive a more comprehensive understanding of the requested data alongside clinical data for similar concepts. Moreover, the use of version mapping enables reproducibility of queries and provides improved data integrity and flexibility by allowing users to retrieve data using query with past versions of clinical coding systems.

As used herein, when an action is “based on” something, this means the action is based at least in part on at least a part of the something.

As used herein, the terms “similarly”, “substantially,” “approximately” and “about” are defined as being largely but not necessarily wholly what is specified (and include wholly what is specified) as understood by one of ordinary skill in the art. In any disclosed embodiment, the term “similarly”, “substantially,” “approximately,” or “about” may be substituted with “within [a percentage] of” what is specified, where the percentage includes 0.1, 1, 5, and 10 percent.

### Overview of Clinical Digital Assistant Architecture

Various types of entities (in this context an entity refers to a person, computing device or system, or software, e.g., users, applications, services such as SaaS, digital assistant systems, database subsystems, etc.) may access a data storage system as described above. In many instances, a heterogeneous data system with disparate data stores that provide different combinations of functionality and data access can be useful to improve application and service workflows and to provide end users with a better experience. A particular example of an environment that can interact with a data storage system to improve functionality and end user experience is in health care environments for accessing clinical data.

Providing healthcare to patients typically requires a healthcare provider (e.g., a physician, nurse professionals, other healthcare professionals, etc.) to repeat a number of common tasks for each patient. For example, regardless of the specific reason for an interaction between a healthcare provider and a patient, or the condition of a given patient, the healthcare provider must typically document the patient interaction. For example, the healthcare provider may record the patient interaction in a subjective, objective, assessment, and plan (SOAP) note, or may enter information gained during the patient interaction into a patient record. The healthcare provider may also engage in various other tasks directly or indirectly related to administering healthcare to the patient, such as requesting additional patient information in the form of charts or images, calling in patient prescriptions, and calendaring future tasks, events, and associated reminders.

Performing such healthcare tasks according to known and commonly used methods can be time consuming. In fact, given the typically high volumes of patient encounters, healthcare providers often spend a considerable portion of their workday documenting patient interactions and associated medical information, which reduces the amount of time available to the healthcare provider to administer actual patient care or perform other more critical tasks. For example, healthcare providers may spend considerable time on a daily basis typing or manually entering patient information into electronic health record (EHR) systems. In addition to being time consuming, this process can be, tedious, and prone to errors such as but not limited to typographical errors, which can result in inaccuracies and inconsistencies in patient records, and can potentially compromise patient safety and the quality of care provided. Traditional EHR systems can also have complex interfaces any may be difficult to navigate, which can increase the time required for healthcare providers to complete such repetitive tasks and generally frustrate the process Traditional EHR system devices may also be cumbersome to operate, and a lack of intercommunication between such devices prevents a healthcare provider from switching between devices while in the process of performing a task even if doing so would be more efficient. These issues may negatively affect patients as well as healthcare providers. For example, patient information may often be retrieved for review or discussion during a patient interaction or recorded during a patient interaction to ensure accuracy. When the process for retrieving or recording such information is inefficient, as is often the case when performed using traditional systems and methods, it can disrupt the natural flow of the patient interaction and may result in a less seamless and less fulfilling experience for the patient. The tedium and time requirements associated with repetitively performing these tasks can also contribute to healthcare provider burnout. Furthermore, such tasks require consistent and accurate access to data, and steps taken to ameliorate and improve task performance (e.g., through automation or otherwise) must also guarantee accurate and consistent access to clinical and/or patient data.

A digital assistant can be implemented using a clinical digital assistant (CDA) framework as described below to improve workflows and capabilities for healthcare providers. The CDA framework interacts with end users and backend systems to enhance healthcare workflows by integrating APIs, multi-modal user interface (UI), and Electronic Health Record (EHR) data sources. End users (e.g., healthcare providers) may interact with the CDA through natural language based conversational experiences. The CDA framework includes generative model (e.g., LLM) based agents that can perform specific functionality (e.g., as defined by a plugin, service level logic, etc.) to provide specialized AI capabilities. In response to a user input, an agent can perform one or more actions including, but not limited, to UI actions that enable conversational interaction against a UI element (e.g., filtering content, adjusting visualization), API actions, and data actions (e.g., retrieving relevant data from a data system). To provide access to internal and external knowledge sources including longitudinal records of a patient and domain-specific knowledge, the CDA framework can include a healthcare semantic index. The healthcare semantic index is a heterogeneous data storage system described above that stores and indexes data, such as patient data, and can enable generative model-based agents to reason across knowledge and data sources through natural language metadata (e.g., as stored in semantic objects) and clinical embeddings (e.g., numeric representations) of unstructured text, images, and discrete data. Access to such data can be important in healthcare settings. For example, a physician performing a chart review may need knowledge about relevant drugs for a condition, interactions between drugs, and interactions between drugs and foods in addition to patient-specific data, such as treatment history.

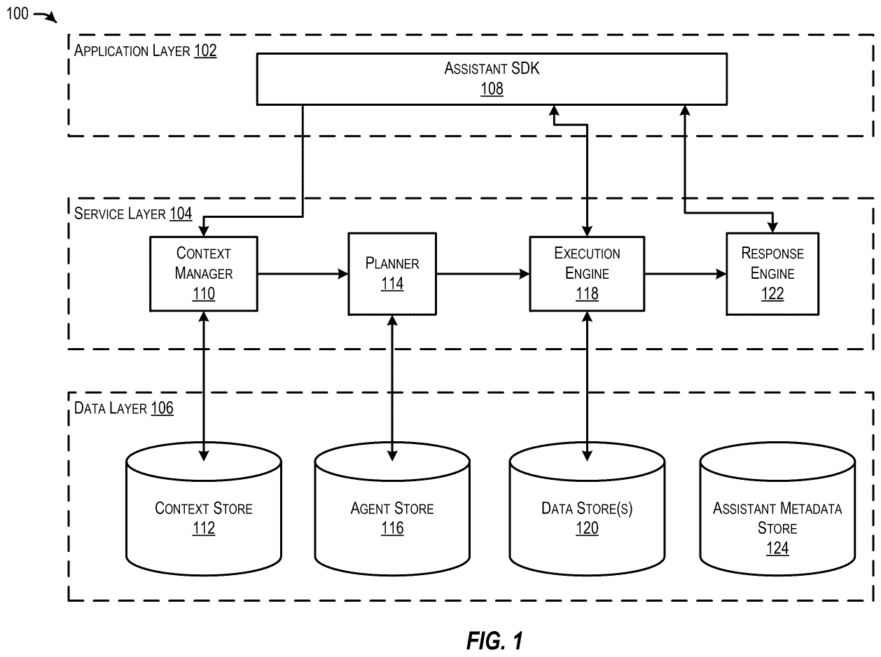

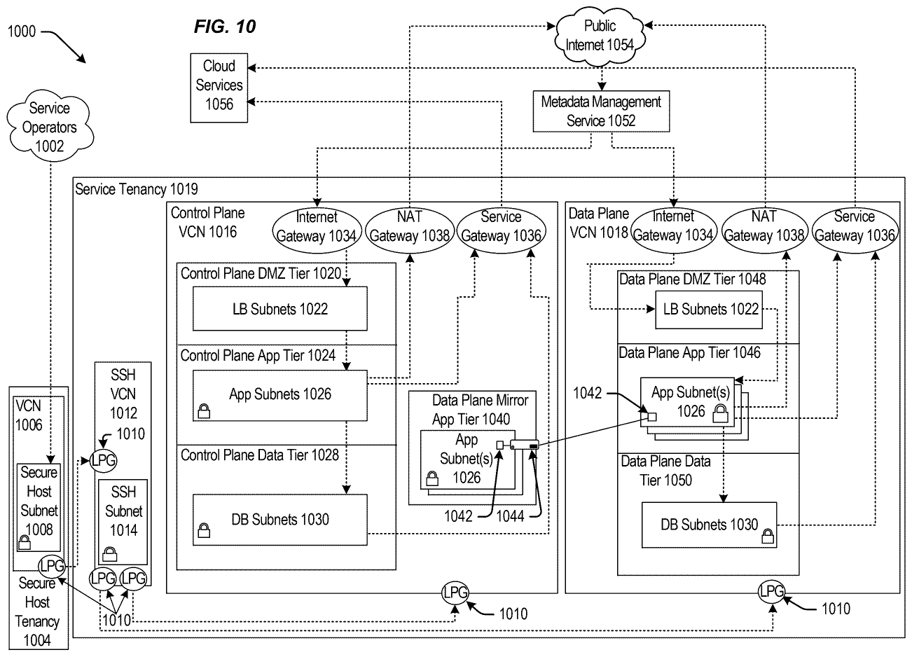

FIG. 1 is an example of an architecture for a computing environment 100 for a clinical digital assistant in accordance with various embodiments. The computing environment 100 can include additional components, fewer components, or different components. In some instances, the computing environment 100 is part of an Infrastructure as a Service (IaaS) cloud service (described in more detail with respect to FIGS. 10-14) and the clinical digital assistant can be implemented as part of the IaaS by leveraging the scalable computing resources and storage capabilities provided by the IaaS provider to process and manage large volumes of data and complex computations.

The computing environment can include various layers including an application layer 102, service layer 104, and data layer 106. Each layer may include components that interact to provide a healthcare workflow as described above. The data layer 106 can be or can include a healthcare semantic index. The application layer 102 can include an assistant software development kit (SDK) 108 that can process user inputs provided by a user through an interface (e.g., a user interface, voice interface, etc.) of an application shell. Examples of user inputs include, but are not limited to, user speech commands, user text commands, user clicks, etc. Additionally or alternatively, the assistant SDK 108 can receive inputs via backend events generated in response to user interactions (e.g., user click events, backend changes, etc.). The assistant SDK 108 can be configured to interact with various components of the service layer 104 (e.g., providing user inputs to the service layer 104, receiving responses from the service layer 104, etc.).

The application layer 102 provides user inputs to a context manager 110 of the service layer 104. The context manager 110 prepares contextual information that can be utilized by components of the service layer 104 to generate a relevant response to the user input and/or user action. The context manager 110 retrieves one or more contexts from a context store 112. A context can act as a holder object for metadata associated with contextual information related to a conversation history, session history, previous executions, etc. The context store 112 can store contexts including, but not limited to, user context, application context, session context, etc. Additionally or alternatively, the context manager 110 may retrieve metadata from an assistant metadata store 124. The assistant metadata store 124 may store metadata for semantic objects and/or plugins that define one or more agents and can be used to identify and select agents and/or actions based on the user input.

The context manager 110 provides contextual information to a planner 114. The planner 114 can be or can utilize one or more generative models (e.g., LLMs or LMMs) fine-tuned to create an execution plan with specified parameters either from a user input (e.g., an utterance), the action performed by the user, the context, or any combination thereof. The execution plan identifies one or more agents and/or one or more actions for the one or more agents to execute in response to the and/or action performed by the user.

The planner 114 can include a retrieval component that retrieves candidate agents and/or actions from the agent store 116. The retrieval component may execute a query on indices of an agent store 116 based on the user input and/or action performed by the user. In some instances, the retrieval component performs a semantic search using words from the user input and/or representative of the action performed by the user. The semantic search uses NLP and optionally machine learning techniques to understand the meaning of the user input and/or action performed by the user and retrieve relevant information from the data layer 106. In contrast to traditional keyword-based searches, which rely on exact matches between the words in the query and the data in the data layer 106, a semantic search takes into account the relationships between words, the context of the query and/or action, synonyms, and other linguistic nuances. This allows the clinical digital assistant to provide more accurate and contextually relevant results, making it more effective in understanding the user's intent in the utterance and/or action performed by the user. The planner 114 can use the candidate agents and/or candidate actions retrieved from the agent store 116 and context determined by the context manager 110 to generate an execution plan listing and/or describing actions that can be executed based on the user input. For example, the planner 114 can determine parameters for the selected action(s) and include the parameters in the execution plan.

The execution plan is transmitted to an execution engine 118 configured to execute the actions of the execution plan. For example, for API actions, the execution engine 118 may execute one or more API calls. For UI actions, the execution engine 118 may populate properties needed to execute the action. For data actions such as knowledge retrieval, the execution engine 118 can execute a query against the data layer 106 (e.g., on one or more data store(s) 120) to retrieve data relevant to the user input. In some examples, to execute a data action, the execution plan can include a semantic search as described that can be executed by the execution engine 118 on the data store(s) to identify relevant information or data (e.g., clinical data related to a certain concept, etc.).

The data layer 106 can be a heterogeneous and disparate data environment as described above. The data layer 106 can include one or more data store(s) 120 that can store patient data (e.g., patient notes, patient discrete data, etc.) and patient agnostic data (e.g., drug information, disease information, drug interaction databases, etc.). The data store(s) 120 can store structured and unstructured data based on the combination of data store(s). For example, the data store(s) 120 can include a relational database, a vector database, an object store, or combinations thereof. One or more of the data store(s) 120 may store clinical embeddings (e.g., in a vector database) to embed knowledge that can be accessed by the service layer 104 and raw data can be enriched by linking information to code-sets (e.g., SNOMED, ICD-10, etc.). Clinical concepts can be stored as semantic objects within the data store(s) 120. The data layer 106 can include data that is kept consistent with a source EHR system. For example, patient data stored in the data layer 106 may be kept consistent with a one or more databases of traditional EHR system through a data ingestion process. As such, changes to patient data made on an external system (e.g., a traditional application used by healthcare providers) can be propagated to the data layer 106 to ensure patient data is accurate irrespective of where the changes are made.

Execution output(s) generated by the execution engine 118 (e.g., data retrieved from the data store(s) 120, API responses, etc.) is transmitted to a response engine 122. The response engine 122 can be or can utilize one or more generative models (e.g., LLMs or LMMs) to generate a response to a user. The response can be a multi-modal response that combines response from different executions into a final response. For example, the response can be text, images, tables, UI elements, action executable by the assistant SDK 108, etc. Response(s) generated by the response engine 122 are transmitted to the assistant SDK 108. The assistant SDK 108 can transmit the response(s) to an application shell to provide the response to the user (e.g., via a user interface, voice interface, etc.).

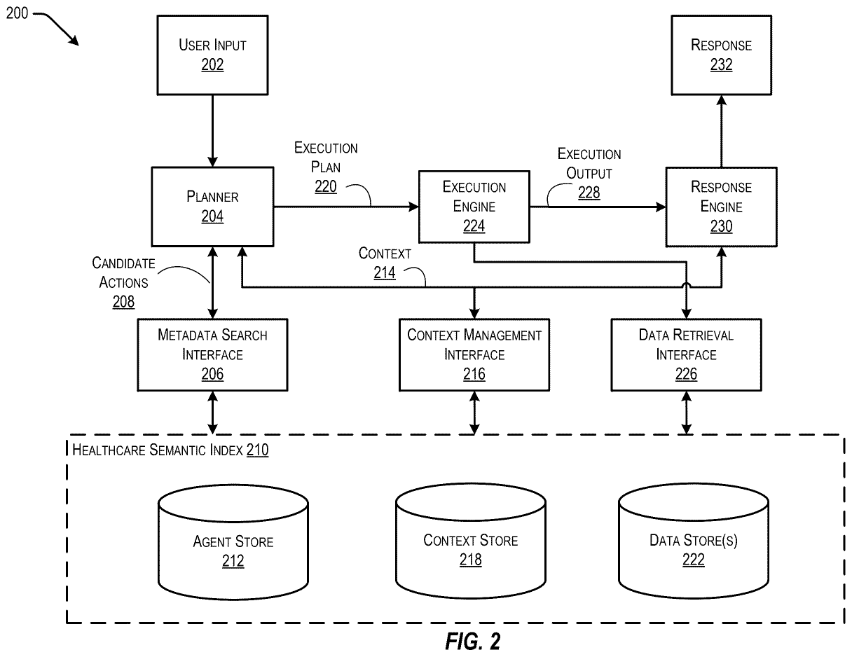

FIG. 2 is a block diagram of a digital assistant runtime flow 200 with components and interfaces into a semantic index, in accordance with various embodiments. As illustrated in FIG. 2, A user input 202 can be provided to a planner 204 (e.g., planner 114 of FIG. 1). The user input 202 can be a natural language utterance, user interface action, programming language query, or other forms of user inputs. In this walkthrough, it is assumed that the user is a healthcare provider interested in knowing medical data of a patient. The healthcare provider provides the following input: Has the patient's total cholesterol level ever been over 180?

Based in the user input 202, the planner 204 accesses a metadata search interface 206 to retrieve appropriate candidate actions 208 from the healthcare semantic index 210 (e.g., data layer 106 of FIG. 1). The candidate actions 208 may be retrieved from an agent store 212 that stores a set of actions associated with one or more agents. Candidate actions 208 can be potential actions determined to meet a confidence threshold for a potential topic related to the user input 202. In some examples, candidate actions 208 can be determined by executing a semantic search on the agent store 212 and identifying actions that satisfy a similarity threshold. Examples of candidate actions 208 include, but are not limited to, UI actions, API actions, data actions, etc. For the above input provided by the healthcare provider, the candidate actions can include actions such as getObservations, getVitals, displayChart, etc. which may each be predefined actions associated with UI changes, data retrieval, API execution, etc.

The planner 204 retrieves context 214 containing contextual information related to the conversation history via a context management interface 216. The context 214 is retrieved from a context store 218 and can include contextual information based on a conversation history and/or session history between the healthcare provider and digital assistant. For example, the context 214 can include a patient id, a current time, previous user utterances, previous responses, etc. For the example of the user input provided by a healthcare provider above, the context 214 can identify the patient referenced in the healthcare provider's input as having a patient identifier value of ‘123’ based on information associated with the session and/or previous interactions (e.g., utterances) between the healthcare provider and the digital assistant.

Based on the retrieved candidate actions 208 and context 214, the planner 204 generates an execution plan 220 that can be executed to answer the healthcare provider's question. The planner 204 selects the most appropriate candidate action of the retrieved candidate actions 208. For the above example, the planner 204 may select the getObservations action to retrieve the patient observations from the healthcare semantic index 210. Additionally, the planner 204 may determine parameters needed to execute the selected action. The parameters can include, for example, an API payload or a query that can be executed on one or more data store(s) 222 of the healthcare semantic index 210. As described with respect to FIG. 1, the planner 204 can be or can make use of one or more LLMs to generate the execution plan 220. In some examples, the candidate actions 208 and context 214 may be provided as a prompt to the planner 204 and/or one or more generative models used by the planner 204 to generate the execution plan 220. For the above example user input 202, the parameters can include a query that can be executed on the healthcare semantic index 210 to retrieve the patient's cholesterol level. A generated execution plan 220 can be as follows:
Action: getObservations
Parameters:
   query: SELECT*FROM Observations WHERE vitalSigns=‘Total Cholesterol’ AND patientID=‘123’ and value >180

The planner 204 provides the execution plan 220 to an execution engine 224 (e.g., execution engine 118 of FIG. 1). The execution engine 224 executes the execution plan 220 to generate an execution output 228. For data actions, the execution engine 224 can execute the execution plan 220 via a data retrieval interface 226. The data retrieval interface 226 can be a programmatic interface for query execution on the healthcare semantic index 210. In some implementations, the data retrieval interface 226 can be or can include one or more API endpoints. Data for a query in the execution plan 220 can be retrieved from one or more data store(s) 222 of the healthcare semantic index 210. The data store(s) 222 can include clinical data stores and may include data stores of various types, including but not limited to relational databases, vector databases, etc.

An execution output 228 generated by the execution engine 224 based on the execution of the execution plan 220 is provided to a response engine 230. The execution output 228 can include data retrieved by execution the execution plan 220, an output of an API call, an action to be performed by an application (e.g., a UI action), references to sources of data and/or outputs, or combinations thereof. The response engine 230 can generate a rich output with appropriate data elements in the output. The response engine can be or can make use of one or more generative models to generate the response 232 that is provided to the user. The response can be an event that is provided to a user, multi-modal response, references, a query result, etc., generated based on the execution output 228 of the execution engine 224. Additionally or alternatively, the response engine 230 can retrieve context 214 (e.g., as retrieved and used by the planner 204) to generate the response 232 with contextual information. For example, the response engine 230 may determine that the name of the patient with patient id ‘123’ as identified above is “Grace” and may include the patient's name in the response 232. A response to the healthcare provider with the above question can be a text and tabular response as follows:

Grace's Total Cholesterol Level was Reported to be Over 180 mg/dL in the Last 2 Lipid Panels.

| Type | Date | Results |
|---|---|---|
| Lipid Panel | Feb. 17, 2024 | Total: 220 mg/dL (elevated) |
| | | HDL: 60 mg/dL (normal) |
| | | LDL: 150 mg/dL (elevated) |
| Lipid Panel | Nov. 17, 2023 | Total: 230 mg/dL (elevated) |
| | | HDL: 60 mg/dL (normal) |
| | | LDL: 160 mg/dL (elevated) |

### Overview of Semantic Index

A Semantic Object Model (SOM) can be an effective way of abstracting data to provide a unified view of data that transcends limitations of individual data storage methods, models, schemas, etc. within a data storage system. A semantic object stored within a data system can represent a particular concept and include various attributes associated with the particular semantic object. In some implementations, the semantic object can include self-describing metadata and/or linked actions for custom operations. The semantic object metadata can be used by a model (e.g., a generative model such as an LLM, etc.) to query the model.

Semantic Index (SI) is a data storage system implementing a Semantic Object Model including disparate and varying types of data stores. SI can store data in a custom way spanning multiple storage systems, abstracting from applications and providing a unified, durable, consistent and powerful data store. As a non-limiting example, SI can be used in healthcare environments (e.g., as described above with respect to the data layer 106 of FIG. 1 and healthcare semantic index of FIG. 2) to improve data accessibility for healthcare professionals and improving patient treatment. Semantic objects within SI can reflect clinical concepts (e.g., patients, treatments, observations, etc.). SI can maintain consistency with a primary source of truth (e.g., an electronic health record (EHR) system of record) to provide patient data related to medical history, diagnoses, etc. Many legacy applications used by healthcare providers rely on prominent platforms supporting conventional EHR systems of record for managing and interfacing with patient and clinical data. For new applications, it can be beneficial to introduce improved semantic techniques to improve access to patient and clinical data. However, for patient records to remain consistent across data platforms and applications, it is important the patient records remain consistent across data models and systems.

In some embodiments, a digital assistant, or chatbot, can interface with the Semantic Index to enable a user to query patient history and data more efficiently. For instance, a digital assistant may be able to query SI using semantic queries generated by a generative model (e.g., a Large Language Model (LLM), etc.). A user may interact with the digital assistant using natural language and then convert the reactions into intelligible queries, such as for clinical questions, etc.

In the interest of clarity of explanation, embodiments of the present disclosure are described in connection with particular data storage systems (e.g., Semantic Index), services (e.g., digital assistants), data models (e.g., Semantic Object Model, relational data models, etc.). However, the embodiments are not limited as such and instead, similarly, and equivalently apply to any data storage system, data models, and services in a multi-data store environment.

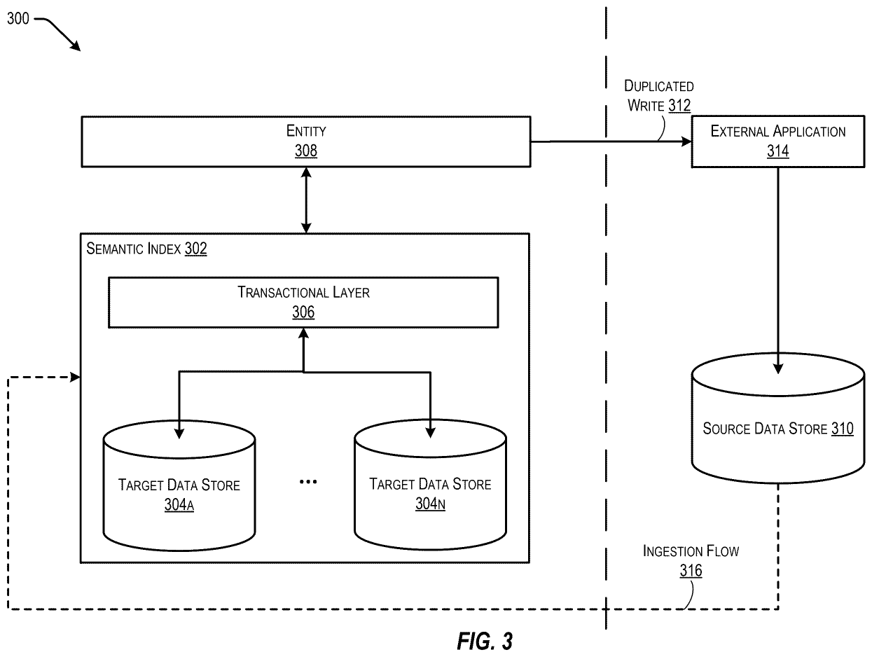

FIG. 3 is a simplified block diagram of an environment 300 of a distributed storage system incorporating Semantic Index. In some instances, the computing environment 300 is part of an Infrastructure as a Service (IaaS) cloud service (as described in more detail with respect to FIGS. 10-14) and semantic index can be implemented as part of the IaaS by leveraging the scalable computing resources and storage capabilities provided by the IaaS provider to process and manage large volumes of data and complex computations. Environment 300 includes Semantic Index (SI) 302 implementing protocols for semantic retrieval of data as described above. While the description of this figure may include various components and processed, it should be understood that additional components, fewer components, or different components as described can be implemented to provide the desired impact.

Semantic Index (SI) 302 can include multiple data stores (e.g., target data store 304a, target data store 304b). In some examples, one or more data stores of target data stores 304a-n are database(s) deployed in a cloud environment using an IaaS cloud service (e.g., as described in more detail with respect to FIGS. 10-14). Each data store within SI 302 may be a different type of data store. For example, target data store 304a can be a vector database (e.g., OpenSearch, Pinecone, etc.) and target data store 304b can be a relational database (e.g., Oracle, MySQL, PostgreSQL, etc.). Additionally or alternatively, Semantic Index 302 can include data stores including, but not limited to, a graph database, NoSQL database, key-value stores, message queues, object stores, etc. Target data stores 304a-n may each contain copies of the same data but provide multiple methods to query and access the data.

While target data stores 304a-n may each be the same and/or different type of data store, each target data store 304a-n may follow the same schema and/or data model. For example, SI 302 can implement the Semantic Object Model as described above, and each target data store 304a-n may implement a schema compatible with the Semantic Object Model. As a particular example, target data store 304b can be a relational database that implements semantic objects as tables within the target data store 304b. Relationships between semantic objects in a relational database may be represented as foreign keys reflecting references to other tables within the target data store 304b.

SI 302 includes a transactional data layer 306 (e.g., data retrieval interface 226 of FIG. 2) that can process queries to SI 302. The transactional layer 306 can support various types of queries, including, but not limited to QDSL, SQL, ingestion from external sources, etc. Additionally or alternatively, the transactional data layer 306 provides a software development kit (SDK) and/or application programming interface (API) that enables an entity 308 (e.g., a user, application, digital assistant, etc.) to interact with Semantic Index 302. For example, the transactional layer 306 includes an API allowing the entity 308 to read and/or write data to SI. The transactional layer 306 can act as an abstraction of the data stored in SI to the entity 308. For example, the entity 308 can call the API to request access to certain data without having knowledge about specific implementations of data models, schemas, and/or data stores within SI 302. Alternatively or additionally, the entity 308 can query SI using a SQL statement, a vector search, or the like. As such, the entity 308 can query and write to SI based on their own internal data models and/or schemas without understanding specifics about the data storage implementations in SI 302. As a particular example, the entity 308 can be a component of a digital assistant system (e.g., planner 204 of FIG. 2) with the capability to receive natural language utterances from a user and determine an execution plan including the execution of one or more programming language queries to retrieve data for addressing and/or responding to the utterances.

In the environment 300 depicted in FIG. 3, writes to SI 302 can occur as a direct write by the entity 308 and/or ingested writes propagated from a source data store 310. The source data store 310 may be a data store externally managed by another organization and/or located in a separate data environment. The source data store 310 may implement a different schema and/or data model than SI 302 and target data stores 304a-n. In some implementations, to maintain consistency between data stored in the target data stores 304a-n and the source data store 310, each direct to SI 302 by the entity 308 may be duplicated to the source data store 310 via a duplicated write 312 provided to an external application 314. The external application 314 may execute the duplicated write 312 on the source data store 310. As an example, in healthcare environments (e.g., as described above with respect to FIGS. 1-2), the source data store 310 can be a database associated with an EHR system. A direct write to SI 302 can include changes to patient data in SI 302. Such changes to patient data are duplicated to the EHR system by providing the duplicated write 312 to the external application 314 (e.g., an application traditionally accessed by a doctor to update patient data) to ensure patient data is consistent.

SI 302 can maintain consistency between the target data stores 304a-n and the source data store 310 via an ingestion flow 316. Data stored in the source data store 310 may be replicated and concurrently stored in the target data stores 304a-n. In some instances, target data stores 304a-n can include data not stored in the source data store 310. For example, SI 302 may store summaries for semantic objects (e.g., stored within a metadata store in SI) that are not compatible with the schema and/or data model implemented by the source data store 310.

Writes to SI can be writes propagated from SI. For example, the external application 314 may execute a direct write on the source database (e.g., a doctor may use PowerChart to update patient data). In eventually consistent models, writes to the source database should be propagated to the target database and, accordingly, such writes can be ingested by SI 302 through the ingestion flow 316. The ingestion flow 316 can be or can include an event stream, change data capture (CDC) system, replication system, or similar that can capture changes in the source database and replicate the changes in a write to SI.

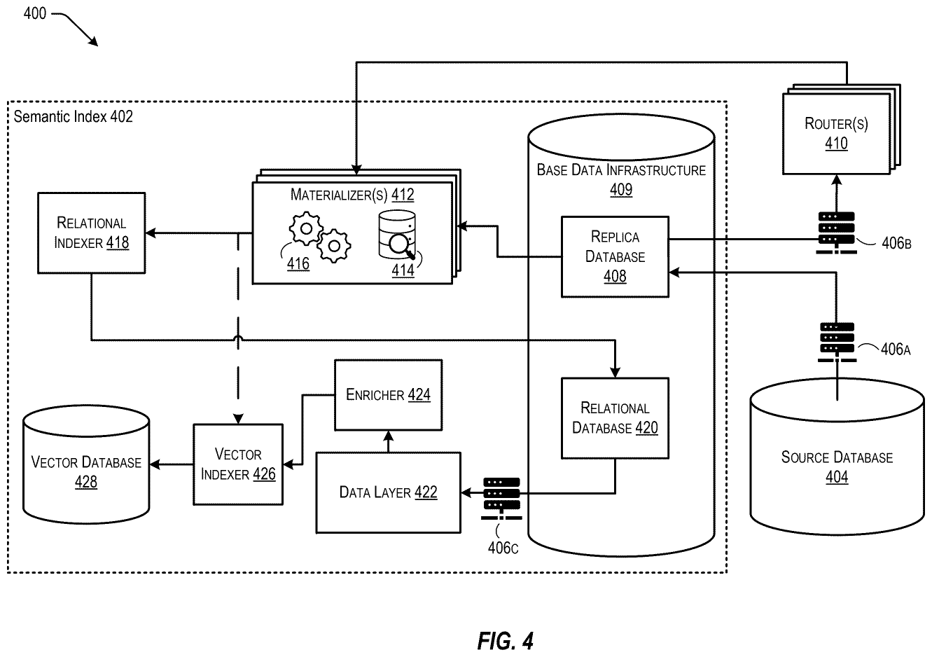

FIG. 4 is an example of an architecture for a computing environment 400 for semantic index implemented with disparate data stores. Certain aspects of FIG. 4 are described with respect to components of the environment described with respect to FIG. 3. As illustrated in FIG. 4, an infrastructure and various services and features can be used to enable the system as described. The following is a detailed walkthrough of an ingestion flow (e.g., ingestion flow 316 of FIG. 3) and the role and responsibility of the components, services, models, and the like of the computing environment 400 within an ingestion flow. In this walkthrough, it is assumed that Semantic Index (SI) 402 is a data storage system that includes data consistent with a source database 404. It is also assumed that any writes to SI 402 are also applied to the source database. In this example, the source database 404 implements a different schema than SI 402 and SI 402 implements a Semantic Object Model.

While the embodiment of computing environment 400 in FIG. 4 illustrates a particular ingestion flow, this is not intended to be limiting and is merely provided to facilitate a better understanding of the role and responsibility of the components, services, models, and the like of the computing environment 400 within the ingestion flow. Some embodiments may include more components than depicted, less components than depicted, or different components than depicted. The ingestion flow, as described, can enable consistent and scalable replication across disparate data stores to enable data synchronization between a source data system and a target data system.

The computing environment 400 includes a source database 404. As described with respect to FIG. 1, the source database 404 can act as a primary source of truth for SI 402. The source database 404 can be a relational database, vector database, NoSQL database, etc. Data stores within semantic index 402 are made consistent with the source database 404. In some implementations, the semantic index 402 includes data not included in the source database 404. As a non-limiting example, the source database 404 is a relational database and acts as an electronic health record (EHR) system of record. The source database 404 may implement a particular schema that is conventionally known.

The source database 404 (e.g., source data store 310 of FIG. 3) can receive a write. For example, a SQL statement may be executed on the source database 404. As described in FIG. 1, the source database can receive the write directly from an external application, or as a duplicated write from a direct write to the Semantic Index 402. By writing the data to the source database, one or more data operations are performed on the source database 404 (e.g., an id is updated, a value is deleted, etc.).

A change data capture (CDC) system 406a (e.g., Kafka, Oracle GoldenGate, Debezium, etc.) may capture data changes in the source database 404 and transmit the data changes to a replica database 408 maintained in semantic index 402. The change data capture system 406a may extract data changes from a transaction log (e.g., redo logs, write-ahead logs, etc.) maintained by the source database 404. The data changes can be transmitted to the replica database 408 as a transaction including one or more data operations (e.g., insertions, deletions, updates, etc.) in the source database 404. In some examples, the data changes can be captured and transmitted as an event stream. The replica database 408 may be a copy of the source database 404 maintained within SI 402 and can serve as the most current known state of the source database 404. The replica database 408 can implement and follow the same schema and/or data model as the source database 404. As such, data changes captured by the CDC system 406a may be executed on the replica database 408 exactly as received. The CDC system 406a can maintain an order of commit of operations executed on the source database 404 and data operations can be executed on the replica database 408 in the order determined by the CDC system 406a.

A second CDC system 406b (e.g., a second Oracle GoldenGate, Debezium, etc.) can capture data changes executed on the replica database 408. The type of CDC system 406b may the same or different as the type of CDC system 406a. The CDC system 406b may extract the data changes from a transaction log maintained by the replica database 408. CDC system 406b packages data changes in the replica database 408 and transmits the data changes to one or more router(s) 410. In some examples, a CDC payload including one or more data operations may be added to a queue associated with the router(s) 410.

The router(s) 410 can be implemented using software only, hardware only, or any combination thereof. The router(s) 410 can be configured to determine semantic objects impacted by data changes in the source database 404 and replica database 408. In some examples, each router 410 may include a mapping of a schema and/or data model implemented by the source database 404 and the schema and/or data model implemented by SI 402. For example, the router(s) 410 may maintain a schema mapping between a table in the source database 404 and semantic objects in SI 402 that consume one or more attributes from the source database 404 table. Accordingly, upon identifying a change to the table in the source database 404, the router(s) 410 may determine the semantic objects impacted by the table update. The router(s) 410 may have a base understanding of attributes and/or fields associated with a particular semantic object. However, each semantic object may include nested structures that the router may be unable to fully and/or accurately map.

The router(s) 410 may not maintain full knowledge of all attributes and nested structures associated with each semantic object in SI 402. For such examples, the router(s) 410 may be configured to identify impacted semantic objects based on table updates, but may not be able to properly construct semantic objects according to the schema implemented by SI 402. The router(s) may invoke one or more materializer(s) 412 to construct the identified impacted semantic objects. The materializer(s) 412 can be implemented with software, hardware, or a combination thereof. Each materializer of the one or more materializer(s) 412 may be configured to construct a particular semantic object. For example, a first materializer may be configured to construct a patient semantic object based on a definition of a patient concept in the semantic model. A second materializer may be configured to construct a treatment semantic object. Upon determining an updated table from the replica database 408 impacts a patient semantic object, the router(s) 410 can invoke the first materializer configured to construct the patient semantic object to generate an updated patient semantic object. The router(s) 410 may invoke multiple materializers by providing each materializer with instructions to construct an updated semantic object. Each materializer of the materializer(s) 412 may construct their respective semantic objects in parallel, sequentially, or any combination thereof.

Each materializer 412 can include a view collector 414 and a finalizer 416. The view collector 414 retrieves current data (e.g., parameters, attributes, data values, etc.) associated with the semantic object based on a known structure of the semantic object. In some examples, the view collector 414 can be a view that presents data from the replica database 408 in a relational and/or JSON format. The finalizer 416 includes software, hardware, or combinations thereof, configured to construct the semantic object using the information retrieved by the view collector 414 from the replica database 408. The semantic object can be subsequently written to the relational database 420. The relational data store can follow the SOM, and each semantic object may be stored in a particular table related to the corresponding semantic object. The semantic object is indexed by a relational data store. In some examples, the finalized semantic object generated by the materializer(s) 412 is provided to a relational indexer 418. The relational indexer 418 may optimize data retrieval and may provide a mechanism for writing the semantic object into its relational shape in the relational database 420. In some examples, the relational indexer 418 may provide pointers to particular rows in the relational database 420 to optimize writes to the relational database 420. Accordingly, semantic objects constructed by the materializer(s) 412 can be written to the relational database 420. The replica database 408 and relational database 420 may be hosted on a base data infrastructure 409. The base data infrastructure 409 may represent the primary source of truth within SI 402, and data stores hosted outside the base data infrastructure 409 may maintain consistency with the base data infrastructure 409.

A third change data capture (CDC) system 406c can capture data changes in the relational database 420. For example, data transactions executed on the relational database 420 to write a finalized semantic object can be reflected in a transaction log associated with the relational database 420. Data changes in the relational database 420 may be associated with an updated semantic object. The CDC system 406c may extract change data from the transaction log associated with relational database 420. The change data can be transmitted to a data layer 422. The data layer 422 may mirror a read-write interface provided by an SDK associated with SI 402 and may orchestrate read-write requests to involve processes such as authorization, persistence, versioning, and event management. In some examples, the data layer 422 may execute processes such as versioning and event management asynchronously.

The change data (e.g., updated semantic object(s) and/or updates associated with one or more semantic object(s)) can be processed by an enricher 424 configured to add context to the data and prepare the data to be stored in the vector database 428. Semantic object data determined from the change data can be vectorized and provided to a vector indexer 426. The vector indexer 426 can provide a mechanism for writing a semantic object in its vectorized shape into the vector database 428. In some examples, the semantic object may be stored as one or more embeddings to capture semantic meaning and relationships across semantic objects.

While the ingestion flow depicted in FIG. 4 depicts a data write executing on the relational database 420 store prior to being converted and indexed to be written to the vector database 428, the finalized semantic object generated by the materializer(s) 412 directly to the vector indexer 426 to be written to the vector database 428. In such ingestion flows, each semantic object may be written in parallel to the relational database 420 and the vector database 428.

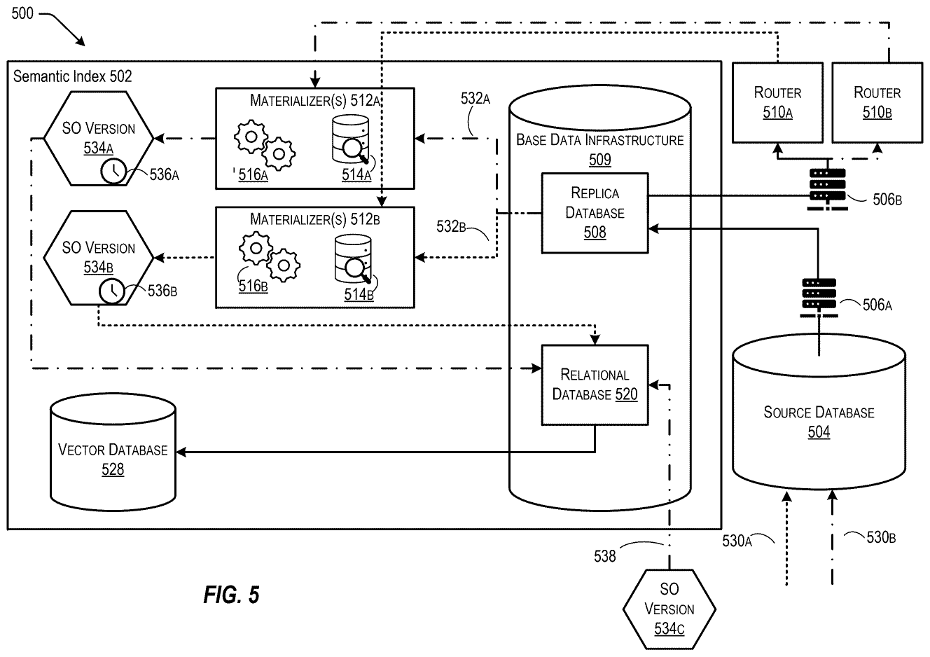

FIG. 5 depicts an ingestion flow 500 implementing watermark generation for replicating multiple data writes from a source system to a target system, in accordance with various embodiments. Certain aspects of FIG. 5 are described with respect to components of the computing environments described with respect to FIG. 4. While the ingestion flow 500 describes watermark generation with respect to writes to the relational database 520 within SI 502, the ingestion flow 500 can include watermark generation for additional target stores within SI 502 such as vector database 528 or any additional target data store not depicted in FIG. 5. Each target data store of SI 502 may maintain distinct sets of watermarks. Additionally or alternatively, source database 504 or other similar source systems may include similar and/or different implementations of watermarking.

At the semantic object level, watermarks can be stored as attributes and/or metadata of a sematic object (e.g., as a timestamp, etc.). A watermark can indicate the freshness of the semantic object and a time up to which the data can be considered accurate. Watermark generation may vary depending on the source of the data write within the data system.

For data writes ingested by SI 502 from a source data system (e.g., source database 504), watermarks can be determined by a materializer configured to generate a particular semantic object. As an example, a source data write 530a is executed on the source database 504. As described with respect to FIG. 4, the source data write 530a is extracted by a CDC system 506a (e.g., CDC system 406a of FIG. 4) and executed on the replica database 508 (e.g., replica database 408 of FIG. 4). The source data write 530a is then processed by a CDC system 506b (e.g., CDC system 406b of FIG. 4) and provided to a router 510a (e.g., router(s) 410 of FIG. 4), which routes the transaction generated by CDC system 506b to a relevant materializer 512a (e.g., materializer(s) 412 of FIG. 4) that can generate an SO version 534a. As used herein, a version of a semantic object can be an instance, data record, etc. of a semantic object that can be stored in a data store (e.g., as generated by a materializer).

The materializer 512a generates the SO version 534a by executing one or more data read(s) 532a on the replica database 508 to retrieve current state information of attributes of the identified semantic object. The materializer 512a can determine a watermark 536a associated with the SO version 534a based on a time at which the one or more data reads 532a were executed. In some examples, the one or more data reads 532a are initiated and/or executed by a view collector 514a (e.g., view collector 414 of FIG. 4) configured to retrieve a current state of data relevant to the semantic object. In some examples (e.g., when the replica database 508 implements a relational data model and/or schema), the view collector 514a may be initialized with one or more predefined Structure Query Language (SQL) queries for creating a view (e.g., virtual table) of the relevant data consumed by the semantic object. The one or more predefined SQL queries for generating a view including data relevant to a particular semantic object can be executed by the view collector 514a, causing one or more data reads 532a to be performed. The watermark 536a can be or can include a timestamp corresponding to the time at which the data read(s) 532a were executed. The timestamp may be based on a time determined by a time determining mechanism of the replica database 508 and/or SI 502. For example, the timestamp may be determined by using a wall clock, logical clock, physical clock, etc. As such, the watermark 536a can reflect the freshness of the data up to the point at which it was read by the materializer 512a.

In some instances, the source database 504 may receive a second source data write 530b that can impact the same semantic object as the source data write 530a. The second source data write 530b may impact the same data within the source database 504 as the first source data write 530a and/or different data within the source database 504. For example, an impacted SO may consume information from a group of tables within the source database 504. The first source data write 530a may update one or more values in a first table within the group of tables. The second source data write 530b may update one or more values in the first table or in a second table within the group of tables. Because the impacted semantic object consumes one or more values from both the first table and the second table, the materializer(s) generate versions of the same semantic object upon receiving the data changes.

Once written to the replica database 508, the first source data write 530a and second source data write 530b may be processed in parallel according to the ingestion flow. For example, a transaction associated with the first source data write 530a may be provided to router 510a by the CDC system 506b and a transaction associated with the second source data write 530b may be provided to a second router 510b. The routers 510a-b may process the respective data writes in sequence, in parallel, or combinations thereof. In some instances, router 510b may finish processing and routing a transaction corresponding to the second source data write 530b before router 510a finishes processing and routing a transaction corresponding to the first source data write 530a despite the first source data write 530a executing on the source database 504 before the second source data write 530b.

Additionally or alternatively, SI 502 may include multiple materializers 512a-512b configured to generate the same semantic object. In some examples, the semantic object generated by the materializers 512a-b may be a semantic object that is identified as receiving frequent updates in the source database 504. The materializers 512a-b may process CDC payloads corresponding to the source data writes in parallel, in sequence, or combinations thereof. Furthermore, the materializer 512a-b may finish processing the payloads in the same order and/or a different order as the processing performed by the routers 510a-b. For example, router 510a may transmit CDC payload(s) to materializer 512a before router 510b transmits CDC payload(s) to materializer 512b, but materializer 512b may generate SO version 534b before materializer 512a generates SO version 534a.

Watermarks determined by the materializers can resolve staleness issues that can be cause by older data writes overwriting new data writes. Each materializer 512a-b generates the respective SO versions 534a-b using information retrieved from replica database 508 from data reads 532a-b. The information retrieved from the replica database 508 can include relevant values for each attribute of the semantic object regardless of whether the value was updated by any particular source data write. As such, the SO versions 534a-b include information about the respective SO from the replica database 508 that is accurate up to the time of the read and can include changes from writes committed after the respective source data writes 530a-b. For example, a third source data write may be executed on the replica database 508 while transactions corresponding to source data writes 530a-b are processed by routers 510a-b and/or before materializers 512a-b retrieve relevant information from the replica database 508 via data reads 532a-b.

The watermarks 536a-b can accordingly reflect the freshness of the semantic object according to the replica database 508 regardless of commit order in the source database 504. For example, the watermark 536b for SO version 534b indicates the SO version is accurate with respect to the source database 504 as of the timestamp reflected by the watermark 536b. The watermark 536a for SO version 534a indicates the SO version is accurate with respect to the source database 504 as of a timestamp reflected by the watermark 536a. SO versions 534a-b can be written to relational database 520 based on a comparison of the watermarks 536a-b and the watermark of the SO as stored in the relational database 520.

Additionally or alternatively, SI 502 can receive a direct data write 538 including changes to a semantic object within a database. The direct data write 538 can be associated with an SO version 534c that may include the same and/or different data as SO versions 534a-b and can result in a stale overwrite depending on commit orders to the source database 504 and to SI 502. SI 502 can generate a placeholder watermark for the SO version 534c corresponding to the direct data write 538. The placeholder watermark may be calculated as a current timestamp incremented by a single unit of time. For example, if the smallest unit of time tracked by SI 502 is a nanosecond, the watermark for SO version 534c may be calculated as the current time incremented by a nanosecond. The SO version 534c can then be written to the relational database 520 according to watermark evaluation of the semantic object version currently stored in the relational database 520. For example, SI 502 can perform a comparison between the watermark of the semantic object as stored in the relational database 520 and the placeholder watermark determined for SO version 534c to determine whether the SO version 534c is associated with fresh data that can safely be written to the relational database 520.

Because SI 502 maintains consistency with the source database 504, one or more duplicated source data write including information associated with SO version 534c can be executed on the source database 504. For example, upon receiving direct data write 538, a duplicated source data write can be generated and executed on the source database 504. The duplicated source data write can subsequently be ingested and generated as described with respect to SO versions 534a-b.

### Querying Diversely Encoded Clinical Data

As described above, differences in implementations of clinical coding systems can make accessing and analyzing clinical data difficult. Accordingly, it can be important for clinical data systems to support query translation to enable interoperability for applications accessing the clinical data store and to reduce user burden when querying the clinical data store. In many instances, users and other entities can also benefit from receiving clinical data related to an exact code mapping between clinical coding systems and from approximate mappings between clinical coding systems when querying using a particular clinical code. With combined exact and approximate matchings, users and other entities can be equipped with clinical data that matches the semantic meaning of the requested clinical code, which may have varying representation as it relates to granularity and organization across clinical coding systems.

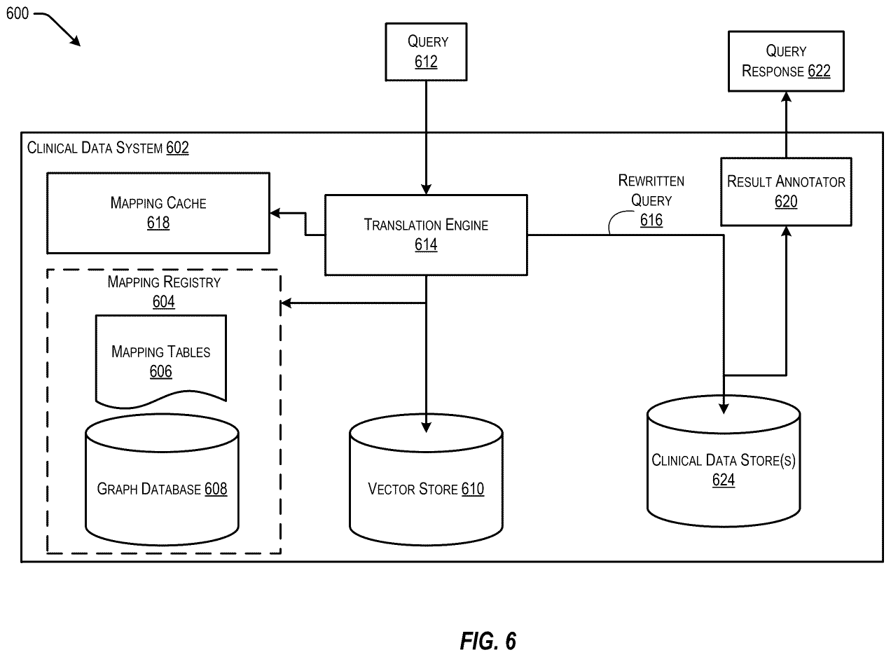

FIG. 6 is a block diagram illustrating an exemplary computing environment 600 implementing semantic translation and expansion of clinical codes, in accordance with various embodiments. The computing environment 600 can include a clinical data system 602 (e.g., SI as described with respect to FIGS. 1-5). The clinical data system 602 can include a one or more clinical data store(s) 624, which can include and interact with clinical data from various sources (e.g., as described with respect to the data layer 106 of FIG. 1 and the healthcare Semantic Index 210 of FIG. 2). In some examples, the clinical data store(s) 624 can store clinical data as semantic object(s) representing medical concepts (e.g., patients, conditions, medications, treatments, etc.). In some examples, semantic objects stored in the clinical data store(s) 624 can include document-based data such as clinical notes stored in a hybrid relational-document database supporting semi-structured data. As described with respect to FIGS. 3-4, clinical data store(s) 624 can maintain consistency with an external source data store (e.g., an EHR system).

Semantic objects stored in the clinical data store(s) 624 can include one or more clinical codes as attributes of the semantic object. In some implementations, the clinical codes can be stored as Codable Concepts (e.g., as defined by the Fast Healthcare Interoperability Resources (FHIR) standard). The clinical data system 602 may maintain an internal clinical coding system that is the same and/or different as a public or external clinical coding system. Accordingly, clinical data system 602 can implement query rewriting to translate clinical codes in queries to equivalent clinical codes of the internal clinical coding system.

The clinical data system 702 includes a mapping registry 604 that stores exact mappings between clinical codes of different clinical coding systems. The mapping registry 604 can store unique identifiers for each coding system that can be used to identify and retrieve mappings between clinical coding systems stored as direct mappings, hierarchical relationships, or combinations thereof. For example, the mapping registry 606 can include one or more mapping tables 606 that store mappings between a clinical code in the internal clinical coding system to one or more equivalent codes in external and/or public clinical coding system (e.g., ICD-10, SNOMED, etc.). The mappings can be one-to-one or one-to-many based on the coding structures and concept granularity of each clinical coding system. The mapping tables 606 can be stored in a dedicated schema with hash-based indexes for fast exact mapping lookups. To generate hash-based index, a hash function can be applied to each clinical code to use as a unique key for the clinical code. The mapping registry 604 can maintain pointers to rows of mapping table 606 where the unique key (e.g., index) occurs to enable retrieval of a code mapping in constant time.

Additionally or alternatively, the mapping registry can include a graph database 608 that stores representations of each coding system and/or mappings between the internal coding system and external coding sources as graphs (e.g., adjacency lists, edge tables, etc.). Hierarchical relationships of codes within a system can be represented as parent nodes (e.g., nodes from which a directed edges originate) and child nodes (e.g., nodes that directed edges point to) in the graph. For example, diabetes mellitus in SNOMED CT is represented by a clinical code (73211009) that includes descendants such as Type 1 diabetes mellitus (46635009). Type 2 diabetes mellitus (44054006), etc. In such examples, broader concepts (e.g., diabetes mellitus) are represented as parent nodes, while more granular concepts (e.g., Type 1 diabetes mellitus and Type 2 diabetes mellitus) are represented as child nodes. Each child node may be parent nodes to additional child nodes list of includes a hierarchy related to diabetes mellitus. Diabetes mellitus has children Type 1 diabetes mellitus and Type 2 diabetes mellitus. Each child has their own children specifying specific types of Type 1 and Type 2 diabetes, respectively. Additionally or alternatively, mappings between clinical coding systems can be included in graphs stored in the graph database 608 through the inclusion of edges representing equivalence and/or similar mappings between nodes of separate coding systems. This can enable traversal of nodes in a graph in the graph database 608 to determine hierarchical relationships between clinical codes in a single coding system and to determine mapping relationships (e.g., one-to-one, one-to-many, many-to-many, etc.) to determine a path from the node of a source clinical code to the node of a target clinical code. Traversal of graphs may be performed by applying graph traversal algorithms. In some examples, shortest-path or depth-limited traversal can be performed (e.g., by using traversal algorithms for weighted hierarchies).

The mapping registry 604 can maintain consistent versions of clinical codes using version metadata. Versions can be tracked not just as versions of clinical codes and definitions for a particular clinical coding system, but also as versions of mappings between clinical coding systems. Accordingly, mapping tables 606 can include mapping tables for each known version of a coding system and can store unique identifiers for each current and past version of a clinical coding system (e.g., “SNOMED_CT_2022”, etc.). The graph database 608 can additionally or alternatively maintain graphs of each mapping version between the internal clinical coding system and the historic and/or current version of the external clinical coding system. As changes to a clinical coding system are made, transactional updates may be committed using ACID transaction to ensure queries continue to maintain access to consistent views of mappings.

In some implementations, versions are maintained in a relational store. Each clinical code value definition can include a version in its definition that can be mapped to other public clinical coding systems. Mappings or coding definitions used to interpret a clinical code or determine equivalent clinical codes in another clinical coding system an include the version used to perform the relevant interpretation or equivalence determination. In such cases, version data may be stored as an additional column in a relational store of the mapping registry 604 and/or clinical data store(s) 624. Such column values for indicating a version of a clinical code can be incremented for clinical code values that are change in new versions of the clinical coding system.

The clinical data system 602 additionally includes a vector store 610 (e.g., vector database 428 of FIG. 4) that stores precomputed vector embeddings for each clinical code across various clinical coding systems. Vector embeddings are numerical representations of data (e.g., clinical codes) that capture semantic and/or structural relationships between items. The precomputed embeddings can be generated using a machine learning model capable of producing dense vector representations in continues vector space (e.g., convolutional neural network, recurrent neural network, transformer model, graph neural network, autoencoder, etc.). As a non-limiting example, a vector embedding for a clinical code can be generated by providing the clinical code and/or a textual description of the clinical code as an input to a generative model pretrained on biomedical textual data (e.g., a clinical language model such as BioBERT). Such models can tokenize the clinical code and textual description and generate token-level embeddings. The token embeddings can be passed through multiple encoder layers (e.g., transformer layers) that refine the embeddings based on the context of surrounding tokens and output a vector for each token. Token-level embeddings can subsequently be pooled and/or aggregated to generate a single vector representing the clinical code. In some implementations, the generated embedding can be fine-tuned for downstream similarity searches. The embeddings can be precomputed such that at runtime (e.g., when a query for semantically similar codes is received), the embedding can be retrieved from the vector store 610 without re-computing the embedding for the clinical code.

The vector store 610 can store vector embeddings for each clinical coding system. In some examples, vector embeddings for different clinical coding systems can be separated into separate collections in the vector store 610. Additionally or alternatively, vector embeddings may be associated with metadata that identifies the associated clinical code and clinical coding system for the embedding. Vector embeddings may also be generated for each version of a clinical coding system (e.g., as stored in the mapping registry 604) to enable semantic searches across versions of a clinical coding set.

A query 612 for clinical data can be received by the clinical data system 602 from a read-write layer (e.g., transactional layer 306 of FIG. 3, data retrieval interface 226 of FIG. 2). The query 604 can include a predicate (e.g., equality expression, etc.) specifying a clinical code. For example, a SQL query can include a predicate such as “WHERE code=<source_clinical_code>” to specify a particular clinical code and a construct (e.g., as listed in Table 1) to specify a coding system. The structure of the query 612 written in SQL may be as follows:

SELECT * FROM <table> WHERE
condition_code = <source_clinical_code>
WITH CODING_SYSTEM <source_coding_system>

The query 612 is provided to a translation engine 614 configured to translate the query 612 to a rewritten query 616 that includes a clinical code used internally by the clinical data system 602 and can be executed on the clinical data store(s) 624 to retrieve clinical data requested in the query 612. The translation engine 614 can be hardware, software, or combinations thereof configured to translate and/or expand queries that can be deployed agnostically to specific implementations of the clinical data system 602. As examples, translation engine 614 can be implemented as a database plugin and/or extension, a query processor of a middleware layer that intercepts and rewrites queries before reaching a clinical data store 624, a microservice for query rewriting, etc. The translation engine 614 can perform direct translations of the query 612 when query 612 is received with a clinical code from a source clinical coding system that does not match the internal clinical coding system implemented by the clinical data system 602.

The translation engine 614 can perform a direct translation by determining one or more equivalent clinical codes (e.g., exact matches) for the source clinical code in the query 612 using mappings stored in the mapping registry 604. For example, the translation engine 614 can perform a hash-based lookup of the clinical code from the mapping tables 606. Additionally or alternatively, the translation engine 614 can perform a traversal of a graph in the graph database 608 as described above. In some examples, the translation engine 614 can retrieve exact mappings from a mapping cache 618 for hot mapping results (e.g., mappings for recently processed clinical codes). In some implementations, the translation engine 614 can be or can include a machine learning model configured to output the exact mappings for a clinical code provided as an input. The machine learning model can be trained on training data that includes input-output pairs including a source clinical code and the corresponding exact mappings. In some instances, the machine learning model can be a generative model (e.g., LLM, LMM) trained and/or fine-tuned using data including clinical codes from various clinical coding systems and optionally definitions for each clinical code. In such instances, the generative model may be prompted to generate a translation for a clinical code according to one or more instructions provided to the model.

If a one-to-one mapping exists between the source clinical code and the exact match, the translation engine replaces the clinical code with the exact match within the query 612 while maintaining the original structure of the query 612 (e.g., replacing an equality clause of WHERE condition_code=<source_clinical_code> with WHERE condition_code=<internal_code>). If the mapping is one-to-many, the translation engine 614 can replace the predicate of the query with a predicate indicating the list of exact matches. For example, the equality clause (e.g., WHERE condition_code=<source_clinical_code>) can be replaced with an IN predicate in SQL (e.g., WHERE condition_code IN (<target_code_1>, <target_code_2>).

Additionally or alternatively, the query 612 can indicate a request for approximate matching. To perform semantic expansion, the translation engine 614 retrieves a pre-computed vector embedding for the source clinical code. The source clinical code embedding may be retrieved by querying the vector store 610 with a known identifier for the clinical code. The translation engine 614 then uses the source clinical code embedding to compute similarity scores (e.g., cosine similarity, Euclidean distance, dot product, etc.) between the source clinical code embedding and the target (e.g., internal) coding system's vector embeddings. The embeddings with the highest similarity scores can be chosen as approximate matches and can be identified as semantically similar. An identifier for each embedding with the highest similarity scores can be determined and used to identify the corresponding internal clinical code.

In some examples, a vector index (e.g., a search structure of the vector store 610) can be stored in specialized vector database plugins to enable fast approximate nearest neighbor search. The source clinical code embedding may be passed to a vector index to perform similarity search on vector embeddings of the internal coding system. The vector index may perform the similarity search by finding the nearest neighbors of the source clinical code embedding in vector space (e.g., via an Approximate Nearest Neighbor (ANN) search). The nearest neighbor search outputs a list of vector embeddings of the ranked based on similarity scores. The internal clinical codes corresponding to the vector embeddings with the highest similarity scores can be determined as approximate matches.

The number of approximate matches can be selected based on an input parameter in the query 612 indicating a number of desired approximate matches (e.g., as listed in Table 1). For example, the query 612 may indicate 5 approximate matches are requested and the clinical codes for the top 5 vector embeddings ranked by similarity score may be selected. Additionally or alternatively, the approximate matches can be chosen based on a similarity score threshold. For example, the similarity score may be computed using cosine similarity and the approximate matches can be selected using all embeddings that exceed a similarity score threshold of 0.9.

When the query 612 includes a semantic expansion construct and/or semantic expansion parameter, the translation engine 614 can perform a two-phase algorithm to generate the rewritten query 616. The translation engine 614 first determines the exact matches as described above (e.g., using the mapping registry 604) and identifies the approximate semantic matches as described above (e.g., by performing a similarity search with vectors in the vector store 610). The translation engine 614 then generates the rewritten query 616 to include both the exact matches and the approximate matches. For example, a SQL query with a predicate containing an equality (e.g., WHERE code=<source_clinical_code>) can be replaced with an IN predicate (e.g., WHERE code IN (<internal_code_1>, . . . , <internal_code_k>). The two sets of matches may be treated with different priorities. The exact matches may be ranked highest with high priority (e.g., primary match conditions), while the approximate matches may be ranked lower (e.g., secondary match conditions). An example rewritten query 616 in SQL can follow the following format:

SELECT * FROM <table> WHERE WHERE condition_code IN
(<internal_code_1>, ...,
<internal_code_k>

The rewritten query 616 may subsequently be executed on the clinical data store(s) 624 to retrieve clinical data. The rewritten query 616 can be executed on a clinical data store of the clinical data store(s) 624 that can optimally return clinical data based on the type of requested data, feasibility of a query, etc. For example, if the query 612 includes a request for clinical notes, the rewritten query 616 may be executed on a clinical data store that supports a hybrid relational-document data model with semi-structured fields. Additionally or alternatively, queries for exact matches (e.g., row-based lookups) may be executed on a relational data store of the clinical data store (e.g., relational database 420 of FIG. 4). Additionally or alternatively, queries for semantic matching (e.g., semantic searches) may be executed on a vector store of the clinical data stores.

The retrieved clinical can include one or more semantic objects with a clinical code attribute corresponding to the clinical codes in the rewritten query 616. For example, for queries on a patient_conditions table, the results can be “Condition” semantic objects that include information about a condition with the requested clinical code. The query results are provided to a result annotator 620 that annotates the results with metadata to generate a query response 622 including the retrieved data and annotated metadata. The annotation generated by the results annotator 620 can capture retrieval provenance and can be or can include metadata associated with the execution of the rewritten query 616. For example, the metadata can include an annotation describing the type of match for an SO (e.g., exact match, approximate match). Additionally or alternatively, for results including approximate matches, the annotation can include a similarity score determined for the clinical code and used to select the clinical code as an approximate match. In some examples, the metadata can include a version mapping and/or embedding model used to identify the matches. The retrieved clinical data can be returned as a list of semantic objects and the annotated metadata can be provided as a list of corresponding metadata. Downstream consumers of the query response 622 (e.g., entity 308 of FIG. 3) can use the annotation to filter or rank results by match confidence or audit specific assets governing retrieval.

An example query response 622 for an exact code match can be as follows:

```
[
{
    “data”: {
      “so_type”: “Condition”,
      “condition_id”: “cond-7f2c9”,
      “patient_id”: “pt-123”,
      “code”: “J45.909”.
      “code_system”: “ICD-10”,
      “code_system_version”: “2025-03”,
      “onset_date”: “2024-11-01”,
      “clinical_status”: “active”,
      “verification_status”: “confirmed”,
      “severity”: “moderate”,
      “provenance”: { “source_system”: “EHR-A”, “last_updated”: “2025-01-12T10:45:22Z” },
      “code_display”: “Unspecified asthma, uncomplicated”
    },
    “annotation”: {
      “match_type”: “Exact”,
      “similarity”: null,
      “mapping_type”: “equivalent”,
      “mapping_version”: “map-icd10-v2025-12”,
      “embedding model version”: null,
      “query_system”: “UMLS”,
      “query_code”: “C0018787”,
      “rewrite_operator”: “translate_only”,
      “explanation”: “UMLS C0018787 → ICD-10 J45.909 (equivalent)”
    }
  }
]
```

An example query response 622 for approximate matches can be as follows:

```
[
  {
    “data”: {
      “so_type”: “Condition”,
      “condition_id”: “cond-91ab4”,
      “patient_id”: “pt-123”,
      “code”: “389145006”,
      “code_system”: “SNOMED”,
      “onset_date”: “2025-02-10”,
      “clinical_status”: “active”
    },
    “annotation”: {
      “match_type”: “Exact”,
      “mapping_version”: “map-snomed-v2025-12”,
      “embedding_model_version”: “emb-clinical-v3.1”,
      “query_system”: “SNOMED”,
      “query_code”: “389145006”,
      “explanation”: “Matched exactly”
    }
  },
  {
    “data”: {
      “so_type”: “Condition”,
      “condition_id”: “cond-77cde”,
      “patient_id”: “pt-789”,
      “code”: “735588005”,
      “code_system”: “SNOMED”,
      “onset_date”: “2024-10-12”,
      “clinical_status”: “active”
    },
    “annotation”: {
      “match_type”: “Approximate”,
      “similarity”: 0.79,
      “mapping_type”: “related”,
      “mapping_version”: “map-snomed-v2025-12”,
      “embedding_model_version”: “emb-clinical-v3.1”,
      “query_system”: “SNOMED”,
      “query_code”: “389145006”,
      “explanation”: “Neighbor selected via
      FUZZY_MATCH (similarity ≥ 0.75)”
    }
  }
]
```

In some instances, the translation engine 614 may be unable to identify any exact code matches and/or approximate matches. In such instances, the clinical data system 602 can implement one or more error handling modes. For example, in a strict mode, an empty result may be returned if no exact and/or approximate matches are found. In a lenient mode, the query result may be returned with the missing exact mapping and/or without expansion. In such instances the result annotator 620 can generate a query response 622 with metadata indicating a missing match.

To expand transparent query translation and expansion, the clinical data system 602 can support new query language constructs. Constructs can refer to query language elements (e.g., expression, structure, etc.) used to modify the results of a query. The clinical data system 602 supports various constructs that enable users or other entities to interact with clinical coding systems in customizable ways. Table 1 includes a list of new constructs that can augment query translation and expansion.

TABLE 1

| Construct Type | Construct Example | Query Example |
|---|---|---|
| Source Coding | WITH CODING_SYSTEM | SELECT * FROM |
| System | <system> | patient_conditions WHERE |
| | | condition_code = ‘C0018787’ |
| | | WITH CODING_SYSTEM |
| | | ‘UMLS’ |
| Target Coding | WITH | SELECT condition_code, |
| System | TARGET_CODING_SYSTEM | description FROM |
| | <system> | patient_conditions WHERE |
| | | condition code = ‘C0018787’ |
| | | WITH CODING_SYSTEM |
| | | ‘UMLS’ AS |
| | | TARGET_CODING_SYSTEM |
| | | ‘ICD10’ |
| Approximate | EXPAND SEMANTICALLY | SELECT * FROM |
| Matching | TOP K | patient_conditions WHERE |
| | | condition_code = ‘C0018787’ |
| | | WITH CODING_SYSTEM |
| | | ‘UMLS’ EXPAND |
| | | SEMANTICALLY TOP 5 |
| Mapping | WITH MAPPING_VERSION | SELECT * FROM |
| Version | <version> | patient_conditions WHERE |
| | | condition_code = ‘C0018787’ |
| | | WITH CODING_SYSTEM |
| | | ‘UMLS’ WITH MAPPING |
| | | VERSION ‘v2025-05’ |
| Hierarchical | EXPAND HIERARCHY TO | SELECT * FROM |
| Expansion | <hierarchy relationship> | patient_conditions WHERE |
| | | condition_code = ‘38341003’ |
| | | WITH CODING SYSTEM |
| | | ‘SNOMED’ EXPAND |
| | | HIERARCHY TO |
| | | DESCENDENTS |

As listed in Table 1, the query 612 can specify a source coding system, which can be used for determining query translations and expansions as described above. The query 612 may also specify approximate matching using the expand semantically construct to request approximate matching with the top K results. Additionally or alternatively, the query 612 can specify a target coding system using a target coding system construct as listed in Table 1. When a target coding system is specified, the result annotator 620 modifies the query results by changing the clinical code(s) of the data records (e.g., semantic objects) from the clinical code of the internal clinical coding system (e.g., as stored in the clinical data store(s) 624) to an equivalent code of the target coding system. The result annotator 620 may access the mapping registry 604 to identify the exact matches of the clinical codes in the target coding system. For example, the result annotator 620 may perform a hash lookup of the mapping tables 606 and/or perform a traversal of a graph in the graph database 608. The target clinical coding system specified in a target coding system construct can be the same and/or different from the source clinical coding system and/or the internal clinical coding system maintained by the clinical data system 602.

Additionally or alternatively, the query 612 can specify a mapping version using a mapping version construct. When the mapping version is specified by the query 612, the translation engine 614 retrieves the exact mappings from the mapping table(s) and/or graphs in the graph database 608 corresponding to the mapping version between the source coding system and/or internal coding system as of a certain time. The mapping version can correspond to a particular version of the source coding system (e.g., a version of a particular month and year, etc.). As shown in the tabular query response 622 above, the result annotator 620 may annotate the query response 622 to include the mapping version metadata corresponding to the indicated mapping version. Additionally or alternatively, the mapping version metadata can include metadata describing the version of the source code used, etc.

Additionally or alternatively, the query 612 can include a request for hierarchical expansion. Hierarchical expansion can include determining broader and/or narrower concepts related to a particular clinical code. To identify the hierarchical expansions, the translation engine 614 can similarly perform a traversal of the graph to identify the parent (e.g., ancestor) and/or child (e.g., descendent) nodes of the identified clinical code. Additionally or alternatively, a table for the clinical coding system may store clinical code relationships indicating parent and/or child nodes of a clinical code. For example, a request to include descendants of a clinical code for diabetes can include clinical codes for Type 1 diabetes and Type 2 diabetes. When generating the rewritten query 616, the translation engine 614 may replace the source clinical code with the determined mappings along with the relevant child and/or parent clinical codes in an IN predicate.

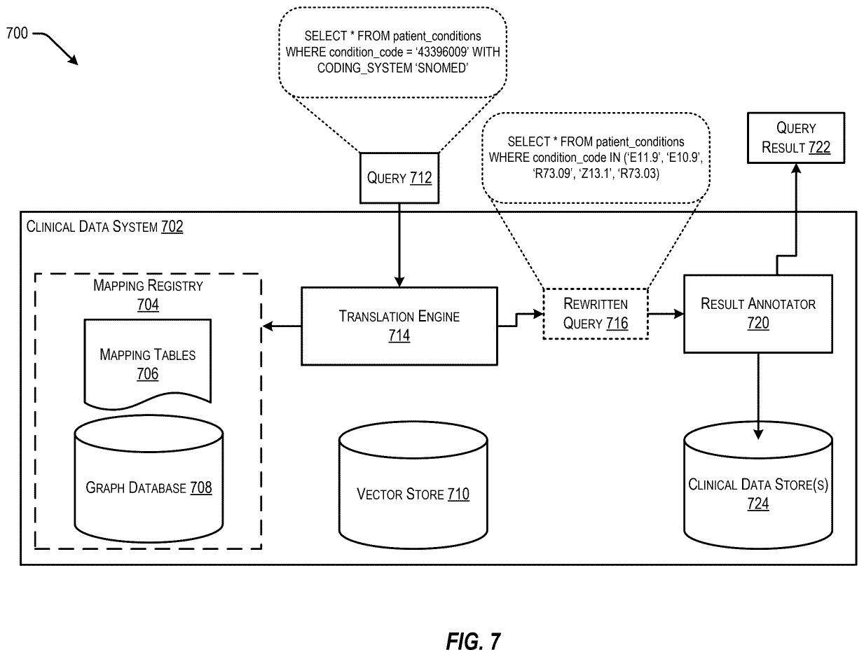

FIG. 7 depicts an example flow 700 for a dynamic direct clinical translation by a clinical data system, in accordance with various embodiments. The flow 700 is not intended to be limiting and is merely provided to facilitate a better understanding of the role and responsibility of components of the clinical data system 702 (e.g., clinical data system 602 of FIG. 6). As a non-limiting example, the clinical data system 702 may implement the ICD-10 clinical coding system internally. As depicted in FIG. 7, the received query 712 specifies the source coding system as UMLS. For example, the query 712 is as follows:

SELECT * FROM patient_conditions WHERE
condition_code = ‘C0018787’ WITH
CODING_SYSTEM ‘UMLS’

Accordingly, the translation engine 714 (e.g., translation engine 614 of FIG. 6) performs a direct translation of the UMLS clinical code C0018787. The translation engine 714 retrieves the exact matches for UMLS clinical code C0018787 from the mapping registry 704. To retrieve the exact matches, the translation engine 714 may perform a lookup using a hash-based index for UMLS clinical code C0018787 from a table containing mappings between UMLS and ICD-10. In some instances, the table may be specified as a current version mapping table and the mapping registry 704 (e.g., mapping registry 604 of FIG. 6) may include mapping tables between ICD-10 and previous versions of UMLS. Additionally or alternatively, the translation engine 714 executes a traversal of a graph in the graph database 708 storing mappings of codes between UMLS and ICD-10 starting at the node for UMLS clinical code C0018787.

The translation engine 714 may retrieve ‘I10’ and ‘I10.9’ as exact matches for C0018787. Accordingly, the translation engine 714 generates a rewritten query 716, as depicted in FIG. 7, that replaces the equality clause of the query 712 (e.g., WHERE condition_code=‘C0018787’) with an IN predicate including the exact matches (e.g., WHERE condition_code IN (‘I10’, ‘I10.9’)). As such, the rewritten query 716 is as follows:
SELECT*FROM patient_conditions WHERE condition_code IN (‘I10’, ‘I10.9’)

The rewritten query 716 is subsequently executed on the clinical data store(s) 724 to retrieve clinical data matching the identified ICD-10 codes. The result annotator 720 can annotate the results to indicate the exact matches and/or the mapping version metadata of the results.

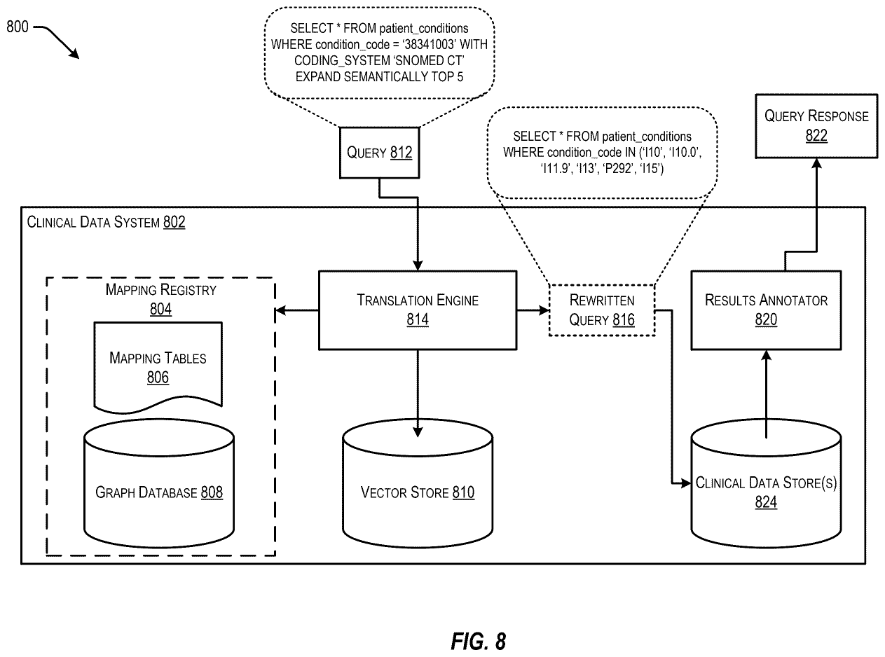

FIG. 8 depicts an example flow 800 for processing a query with dynamic translation and semantic expansion, in accordance with various embodiments. The flow 800 is not intended to be limiting and is merely provided to facilitate a better understanding of the role and responsibility of components of the clinical data system 802 (e.g., clinical data system 602 of FIG. 6).

As a non-limiting example, a user interacting with a clinical digital assistant system (e.g., as described with respect to FIGS. 1-2) may provide the following natural language utterance: I'd like to know more about hypertension. A planner (e.g., planner 204 of FIG. 2) of the clinical digital assistant system may use contextual information based on previous sessions with the user to determine that information about general hypertension may not be adequate information to provide the user. As such, the planner may generate query 812 to include a semantic expansion parameter (“EXPAND SEMANTICALLY TOP 5”) indicating a request for approximate matching with the five most similar clinical codes. The planner may be equipped The user may primarily interact with the SNOMED CT clinical coding system and the planner may according generate the query 812 using the SNOMED CT clinical code for the general code for hypertension (38341003). The query 812 is executed on the clinical data system 802 (e.g., clinical data system 602 of FIG. 6) by an execution engine and sent to a translation engine 814 (e.g., translation engine 614 of FIG. 6).

In this example, the internal clinical coding system maintained by the clinical data system 802 is ICD-10. The translation engine 814 uses the relevant hash-based index for SNOMED CT clinical code 38341003 to retrieve the exact mapping from mapping tables 806 (e.g., mapping tables 606 of FIG. 6) and/or performs a traversal of a graph model in graph database 808 connecting the SNOMED CT and ICD-10 by starting at a node for SNOMED code 38341003. The translation engine 814 may retrieve ICD-10 clinical code I10 (Essential (primary) hypertension) as the exact match.

Additionally, the translation engine 814 retrieves a pre-computed vector embedding for SNOMED CT clinical code 38341003 from vector store 810. The translation engine 814 sends the embedding for SNOMED CT clinical code 38341003 to a vector index of vector store 810 to perform a similarity search for similar codes in ICD-10. The vector index executes an approximate nearest neighbors search on embeddings of ICD-10 and computes a similarity score (e.g., cosine similarity) between the embedding for SNOMED CT clinical code 38341003 and embeddings ICD-10. Among the embeddings for ICD-10, the embeddings with the five highest similarity scores are selected as the approximate matches. In this example, the ICD-10 clinical codes I10.0 (Hypertensive heart disease with heart failure), I11.9 (Hypertensive heart disease without heart failure), I13 (Hypertensive heart and chronic kidney failure), P292 (Neonatal hypertension), and I15 (Secondary hypertension).

The translation engine 814 generates a rewritten query 816 by replacing the equality clause with an IN predicate including both the exact match and the approximate matches. As such, the rewritten query 816 is as follows:

SELECT * FROM patient_conditions WHERE condition_code IN
(‘I10’, ‘I10.0’, ‘I11.9’, ‘I13’,
‘P292’, ‘I15’)

The rewritten query 816 is executed on the clinical data store(s) and semantic objects related to hypertension and associated with the identified I10 clinical codes are retrieved. The results annotator 820 annotates the results to generate a query response 822 with metadata indicating semantic objects with an I10 clinical code are exact matches, while semantic objects with ‘I10.0’, ‘I11.9’, ‘I13’, ‘P292’, and ‘I15’ clinical codes are approximate matches. The semantic objects for the approximate matches are also annotated with metadata with the respective similarity scores computed using the respective vector embeddings for each clinical code.

The retrieved semantic objects in the query response 822 can include various clinical information about related to each clinical codes including clinical notes stored as semi-structured data. A response engine (e.g., response engine 230 of FIG. 2) the user with clinical notes from the exact match semantic objects and the semantic objects annotated with the highest similarity scores in the metadata. Additionally, the response engine may generate brief summary of each clinical note.

### Illustrative Methods

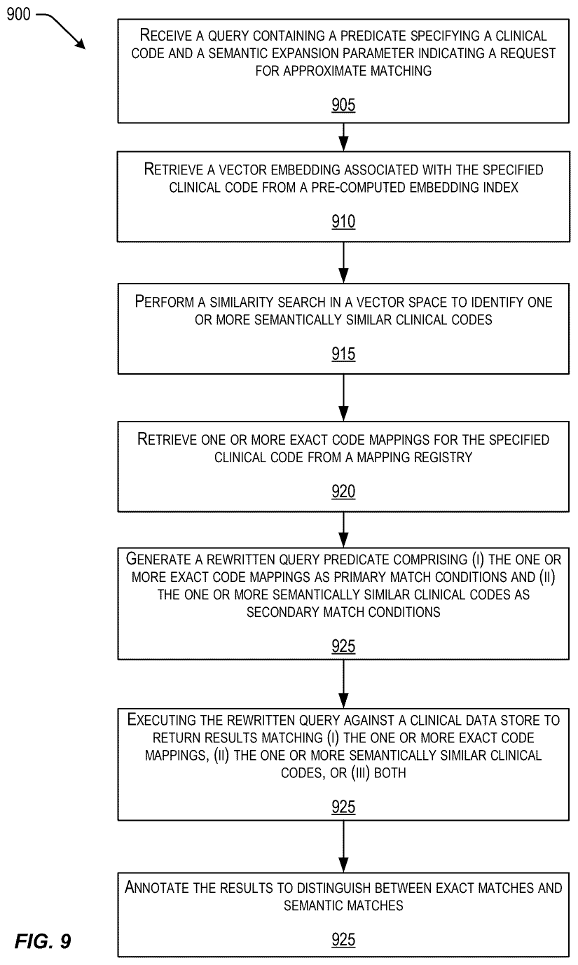

FIG. 9 is a flowchart of a process 900 for executing queries with semantic code expansion in a clinical data system in accordance with various embodiments. The processing depicted in FIG. 9 may be implemented in software (e.g., code, instructions, program) executed by one or more processing units (e.g., processors, cores) of the respective systems, hardware, or combinations thereof. The software may be stored on a non-transitory storage medium (e.g., on a memory device). The process presented in FIG. 9 and described below is intended to be illustrative and non-limiting. Although FIG. 9 illustrates the various processing steps occurring in a particular sequence or order, this is not intended to be limiting. In certain alternative embodiments, the steps may be performed in some different order or some steps may also be performed at least partially in parallel. In certain embodiments, the processing depicted in FIG. 9 may be performed by one or more of the components, computing devices, services, or the like, such as a data storage system, semantic index, etc., illustrated and described with respect to FIGS. 1-9 and FIGS. 10-14.

At step 905, a query (e.g., query 612 of FIG. 6) containing a predicate specifying a clinical code and a semantic expansion parameter indicating a request for approximate matching is received. In some examples, the query can include one or more constructs indicating a coding system associated with the clinical code and/or a target coding system for approximate matching. In some examples, the semantic expansion parameter indicates a type of semantic expansion and/or a number of candidate codes. In some examples, the query may be generated based on a natural language utterance provided by a user (e.g., by planner 204 of FIG. 2).

In some examples, the query indicates a specified version of the clinical code. The specified version may correspond to a historical value of the clinical code. Retrieving the one or more exact mapping can include retrieving one or more exact code mappings associated with the specified version of the clinical code.

At step 910, a vector embedding associated with the specified clinical code is retrieved from a pre-computed embedding index. The retrieved vector embedding may be associated with the specified version of the clinical code.

At step 915, a similarity search in vector space is performed based on the vector embedding to identify one or more semantically similar clinical codes. In some examples, performing the similarity search includes computing a set of similarity scores. Each similarity score of the set of similarity scores is computed between the retrieved vector embedding and a vector embedding of a candidate code. The one or more semantically similar clinical codes can be selected by identifying one or more similarity scores of the set of similarity scores that exceed a threshold.

At step 920, one or more exact code mapping for the specified clinical code can be retrieved from a mapping registry (e.g., mapping registry 604 of FIG. 6). In some examples, the mapping registry can include a mapping table (e.g., mapping tables 606 of FIG. 6) including hash-based indexes. The one or more exact code mappings can be retrieved by performing a point lookup based on a hash index of the clinical code.

In some examples, the mapping registry includes a hierarchical graph stored in a graph database (e.g., graph database 608 of FIG. 6). The one or more exact code mappings can be retrieved by performing a traversal of the hierarchical graph based on the clinical code and one or more coding system to identify the one or more exact code mappings.

At step 925, a rewritten query predicate including (i) the one or more exact code mappings as primary match conditions and (ii) the one or more semantically similar clinical codes as secondary match conditions.

At step 930, the rewritten query is executed against a clinical data store to return results matching (i) the one or more exact code mappings, (ii) the one or more semantically similar clinical codes, or (iii) both. In some examples, the clinical data system includes a plurality of clinical data stores. The results matching the exact code mappings may be retrieved from a relational data store of the plurality of clinical data stores. The results matching the semantically similar clinical codes may be retrieved from a vector data store of the plurality of data stores.

At step 935, the results are annotated to distinguish between exact matches and semantic matches. In some examples, a relevance score can be determined for each result matching the one or more semantically similar codes. The results may be annotated with the relevance score for each semantic match. In some examples, the results can be annotated with mapping version metadata.

### Examples of Architectures for Implementing Cloud Infrastructures

As noted above, infrastructure as a service (IaaS) is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (example services include billing software, monitoring software, logging software, load balancing software, clustering software, etc.). Thus, as these services may be policy-driven, IaaS users may be able to implement policies to drive load balancing to maintain application availability and performance.

In some instances, IaaS customers may access resources and services through a wide area network (WAN), such as the Internet, and can use the cloud provider's services to install the remaining elements of an application stack. For example, the user can log in to the IaaS platform to create virtual machines (VMs), install operating systems (OSs) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand)) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different challenges for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (VPCs) (e.g., a potentially on-demand pool of configurable and/or shared computing resources), also known as a core network. In some examples, there may also be one or more inbound/outbound traffic group rules provisioned to define how the inbound and/or outbound traffic of the network will be set up and one or more virtual machines (VMs). Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

FIG. 10 is a block diagram 1000 illustrating an example pattern of an IaaS architecture, according to at least one embodiment. Service operators 1002 can be communicatively coupled to a secure host tenancy 1004 that can include a virtual cloud network (VCN) 1006 and a secure host subnet 1008. In some examples, the service operators 1002 may be using one or more client computing devices, which may be portable handheld devices (e.g., an iPhone®, cellular telephone, an iPad®, computing tablet, a personal digital assistant (PDA)) or wearable devices (e.g., a Google Glass® head mounted display), running software such as Microsoft Windows Mobile®, and/or a variety of mobile operating systems such as iOS, Windows Phone, Android, BlackBerry 8, Palm OS, and the like, and being Internet, e-mail, short message service (SMS), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 1006 and/or the Internet.

The VCN 1006 can include a local peering gateway (LPG) 1010 that can be communicatively coupled to a secure shell (SSH) VCN 1012 via an LPG 1010 contained in the SSH VCN 1012. The SSH VCN 1012 can include an SSH subnet 1014, and the SSH VCN 1012 can be communicatively coupled to a control plane VCN 1016 via the LPG 1010 contained in the control plane VCN 1016. Also, the SSH VCN 1012 can be communicatively coupled to a data plane VCN 1018 via an LPG 1010. The control plane VCN 1016 and the data plane VCN 1018 can be contained in a service tenancy 1019 that can be owned and/or operated by the IaaS provider.

The control plane VCN 1016 can include a control plane demilitarized zone (DMZ) tier 1020 that acts as a perimeter network (e.g., portions of a corporate network between the corporate intranet and external networks). The DMZ-based servers may have restricted responsibilities and help keep breaches contained. Additionally, the DMZ tier 1020 can include one or more load balancer (LB) subnet(s) 1022, a control plane app tier 1024 that can include app subnet(s) 1026, a control plane data tier 1028 that can include database (DB) subnet(s) 1030 (e.g., frontend DB subnet(s) and/or backend DB subnet(s)). The LB subnet(s) 1022 contained in the control plane DMZ tier 1020 can be communicatively coupled to the app subnet(s) 1026 contained in the control plane app tier 1024 and an Internet gateway 1034 that can be contained in the control plane VCN 1016, and the app subnet(s) 1026 can be communicatively coupled to the DB subnet(s) 1030 contained in the control plane data tier 1028 and a service gateway 1036 and a network address translation (NAT) gateway 1038. The control plane VCN 1016 can include the service gateway 1036 and the NAT gateway 1038.

The control plane VCN 1016 can include a data plane mirror app tier 1040 that can include app subnet(s) 1026. The app subnet(s) 1026 contained in the data plane mirror app tier 1040 can include a virtual network interface controller (VNIC) 1042 that can execute a compute instance 1044. The compute instance 1044 can communicatively couple the app subnet(s) 1026 of the data plane mirror app tier 1040 to app subnet(s) 1026 that can be contained in a data plane app tier 1046.

The data plane VCN 1018 can include the data plane app tier 1046, a data plane DMZ tier 1048, and a data plane data tier 1050. The data plane DMZ tier 1048 can include LB subnet(s) 1022 that can be communicatively coupled to the app subnet(s) 1026 of the data plane app tier 1046 and the Internet gateway 1034 of the data plane VCN 1018. The app subnet(s) 1026 can be communicatively coupled to the service gateway 1036 of the data plane VCN 1018 and the NAT gateway 1038 of the data plane VCN 1018. The data plane data tier 1050 can also include the DB subnet(s) 1030 that can be communicatively coupled to the app subnet(s) 1026 of the data plane app tier 1046.

The Internet gateway 1034 of the control plane VCN 1016 and of the data plane VCN 1018 can be communicatively coupled to a metadata management service 1052 that can be communicatively coupled to public Internet 1054. Public Internet 1054 can be communicatively coupled to the NAT gateway 1038 of the control plane VCN 1016 and of the data plane VCN 1018. The service gateway 1036 of the control plane VCN 1016 and of the data plane VCN 1018 can be communicatively coupled to cloud services 1056.

In some examples, the service gateway 1036 of the control plane VCN 1016 or of the data plane VCN 1018 can make application programming interface (API) calls to cloud services 1056 without going through public Internet 1054. The API calls to cloud services 1056 from the service gateway 1036 can be one-way: the service gateway 1036 can make API calls to cloud services 1056, and cloud services 1056 can send requested data to the service gateway 1036. But, cloud services 1056 may not initiate API calls to the service gateway 1036.

In some examples, the secure host tenancy 1004 can be directly connected to the service tenancy 1019, which may be otherwise isolated. The secure host subnet 1008 can communicate with the SSH subnet 1014 through an LPG 1010 that may enable two-way communication over an otherwise isolated system. Connecting the secure host subnet 1008 to the SSH subnet 1014 may give the secure host subnet 1008 access to other entities within the service tenancy 1019.

The control plane VCN 1016 may allow users of the service tenancy 1019 to set up or otherwise provision desired resources. Desired resources provisioned in the control plane VCN 1016 may be deployed or otherwise used in the data plane VCN 1018. In some examples, the control plane VCN 1016 can be isolated from the data plane VCN 1018, and the data plane mirror app tier 1040 of the control plane VCN 1016 can communicate with the data plane app tier 1046 of the data plane VCN 1018 via VNICs 1042 that can be contained in the data plane mirror app tier 1040 and the data plane app tier 1046.

In some examples, users of the system, or customers, can make requests, for example create, read, update, or delete (CRUD) operations, through public Internet 1054 that can communicate the requests to the metadata management service 1052. The metadata management service 1052 can communicate the request to the control plane VCN 1016 through the Internet gateway 1034. The request can be received by the LB subnet(s) 1022 contained in the control plane DMZ tier 1020. The LB subnet(s) 1022 may determine that the request is valid, and in response to this determination, the LB subnet(s) 1022 can transmit the request to app subnet(s) 1026 contained in the control plane app tier 1024. If the request is validated and requires a call to public Internet 1054, the call to public Internet 1054 may be transmitted to the NAT gateway 1038 that can make the call to public Internet 1054. Metadata that may be desired to be stored by the request can be stored in the DB subnet(s) 1030.

In some examples, the data plane mirror app tier 1040 can facilitate direct communication between the control plane VCN 1016 and the data plane VCN 1018. For example, changes, updates, or other suitable modifications to configuration may be desired to be applied to the resources contained in the data plane VCN 1018. Via a VNIC 1042, the control plane VCN 1016 can directly communicate with, and can thereby execute the changes, updates, or other suitable modifications to configuration to, resources contained in the data plane VCN 1018.

In some embodiments, the control plane VCN 1016 and the data plane VCN 1018 can be contained in the service tenancy 1019. In this case, the user, or the customer, of the system may not own or operate either the control plane VCN 1016 or the data plane VCN 1018. Instead, the IaaS provider may own or operate the control plane VCN 1016 and the data plane VCN 1018, both of which may be contained in the service tenancy 1019. This embodiment can enable isolation of networks that may prevent users or customers from interacting with other users', or other customers', resources. Also, this embodiment may allow users or customers of the system to store databases privately without needing to rely on public Internet 1054, which may not have a desired level of threat prevention, for storage.

In other embodiments, the LB subnet(s) 1022 contained in the control plane VCN 1016 can be configured to receive a signal from the service gateway 1036. In this embodiment, the control plane VCN 1016 and the data plane VCN 1018 may be configured to be called by a customer of the IaaS provider without calling public Internet 1054. Customers of the IaaS provider may desire this embodiment since database(s) that the customers use may be controlled by the IaaS provider and may be stored on the service tenancy 1019, which may be isolated from public Internet 1054.

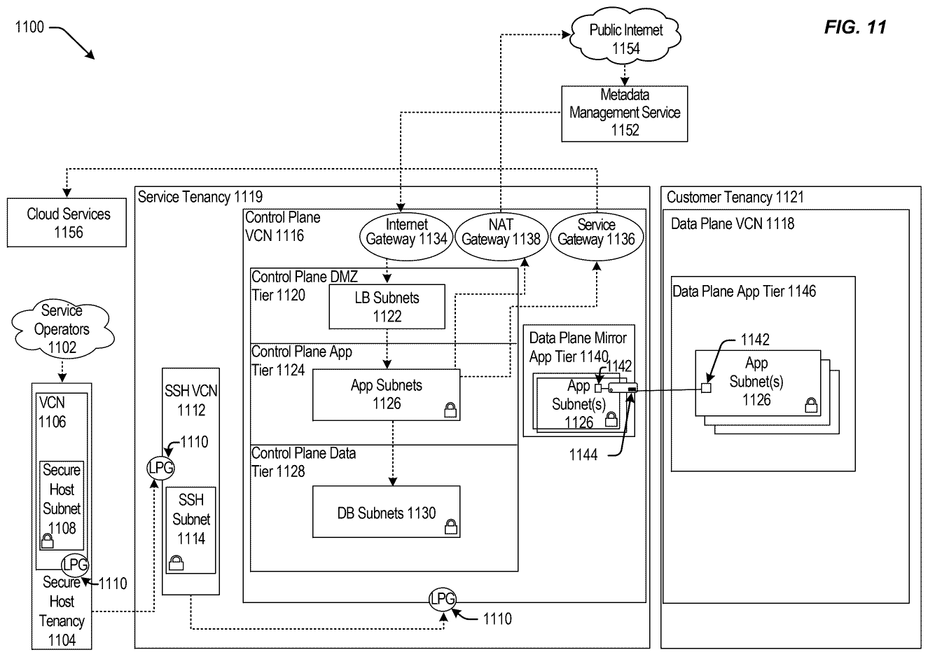

FIG. 11 is a block diagram 1100 illustrating another example pattern of an IaaS architecture, according to at least one embodiment. Service operators 1102 (e.g., service operators 1002 of FIG. 10) can be communicatively coupled to a secure host tenancy 1104 (e.g., the secure host tenancy 1004 of FIG. 10) that can include a virtual cloud network (VCN) 1106 (e.g., the VCN 1006 of FIG. 10) and a secure host subnet 1108 (e.g., the secure host subnet 1008 of FIG. 10). The VCN 1106 can include a local peering gateway (LPG) 1110 (e.g., the LPG 1010 of FIG. 10) that can be communicatively coupled to a secure shell (SSH) VCN 1112 (e.g., the SSH VCN 1012 of FIG. 10) via an LPG 1010 contained in the SSH VCN 1112. The SSH VCN 1112 can include an SSH subnet 1114 (e.g., the SSH subnet 1014 of FIG. 10), and the SSH VCN 1112 can be communicatively coupled to a control plane VCN 1116 (e.g., the control plane VCN 1016 of FIG. 10) via an LPG 1110 contained in the control plane VCN 1116. The control plane VCN 1116 can be contained in a service tenancy 1119 (e.g., the service tenancy 1019 of FIG. 10), and the data plane VCN 1118 (e.g., the data plane VCN 1018 of FIG. 10) can be contained in a customer tenancy 1121 that may be owned or operated by users, or customers, of the system.

The control plane VCN 1116 can include a control plane DMZ tier 1120 (e.g., the control plane DMZ tier 1020 of FIG. 10) that can include LB subnet(s) 1122 (e.g., LB subnet(s) 1022 of FIG. 10), a control plane app tier 1124 (e.g., the control plane app tier 1024 of FIG. 10) that can include app subnet(s) 1126 (e.g., app subnet(s) 1026 of FIG. 10), a control plane data tier 1128 (e.g., the control plane data tier 1028 of FIG. 10) that can include database (DB) subnet(s) 1130 (e.g., similar to DB subnet(s) 1030 of FIG. 10). The LB subnet(s) 1122 contained in the control plane DMZ tier 1120 can be communicatively coupled to the app subnet(s) 1126 contained in the control plane app tier 1124 and an Internet gateway 1134 (e.g., the Internet gateway 1034 of FIG. 10) that can be contained in the control plane VCN 1116, and the app subnet(s) 1126 can be communicatively coupled to the DB subnet(s) 1130 contained in the control plane data tier 1128 and a service gateway 1136 (e.g., the service gateway 1036 of FIG. 10) and a network address translation (NAT) gateway 1138 (e.g., the NAT gateway 1038 of FIG. 10). The control plane VCN 1116 can include the service gateway 1136 and the NAT gateway 1138.

The control plane VCN 1116 can include a data plane mirror app tier 1140 (e.g., the data plane mirror app tier 1040 of FIG. 10) that can include app subnet(s) 1126. The app subnet(s) 1126 contained in the data plane mirror app tier 1140 can include a virtual network interface controller (VNIC) 1142 (e.g., the VNIC of 1042) that can execute a compute instance 1144 (e.g., similar to the compute instance 1044 of FIG. 10). The compute instance 1144 can facilitate communication between the app subnet(s) 1126 of the data plane mirror app tier 1140 and the app subnet(s) 1126 that can be contained in a data plane app tier 1146 (e.g., the data plane app tier 1046 of FIG. 10) via the VNIC 1142 contained in the data plane mirror app tier 1140 and the VNIC 1142 contained in the data plane app tier 1146.

The Internet gateway 1134 contained in the control plane VCN 1116 can be communicatively coupled to a metadata management service 1152 (e.g., the metadata management service 1052 of FIG. 10) that can be communicatively coupled to public Internet 1154 (e.g., public Internet 1054 of FIG. 10). Public Internet 1154 can be communicatively coupled to the NAT gateway 1138 contained in the control plane VCN 1116. The service gateway 1136 contained in the control plane VCN 1116 can be communicatively coupled to cloud services 1156 (e.g., cloud services 1056 of FIG. 10).

In some examples, the data plane VCN 1118 can be contained in the customer tenancy 1121. In this case, the IaaS provider may provide the control plane VCN 1116 for each customer, and the IaaS provider may, for each customer, set up a unique compute instance 1144 that is contained in the service tenancy 1119. Each compute instance 1144 may allow communication between the control plane VCN 1116, contained in the service tenancy 1119, and the data plane VCN 1118 that is contained in the customer tenancy 1121. The compute instance 1144 may allow resources, that are provisioned in the control plane VCN 1116 that is contained in the service tenancy 1119, to be deployed or otherwise used in the data plane VCN 1118 that is contained in the customer tenancy 1121.

In other examples, the customer of the IaaS provider may have databases that live in the customer tenancy 1121. In this example, the control plane VCN 1116 can include the data plane mirror app tier 1140 that can include app subnet(s) 1126. The data plane mirror app tier 1140 can reside in the data plane VCN 1118, but the data plane mirror app tier 1140 may not live in the data plane VCN 1118. That is, the data plane mirror app tier 1140 may have access to the customer tenancy 1121, but the data plane mirror app tier 1140 may not exist in the data plane VCN 1118 or be owned or operated by the customer of the IaaS provider. The data plane mirror app tier 1140 may be configured to make calls to the data plane VCN 1118 but may not be configured to make calls to any entity contained in the control plane VCN 1116. The customer may desire to deploy or otherwise use resources in the data plane VCN 1118 that are provisioned in the control plane VCN 1116, and the data plane mirror app tier 1140 can facilitate the desired deployment, or other usage of resources, of the customer.

In some embodiments, the customer of the IaaS provider can apply filters to the data plane VCN 1118. In this embodiment, the customer can determine what the data plane VCN 1118 can access, and the customer may restrict access to public Internet 1154 from the data plane VCN 1118. The IaaS provider may not be able to apply filters or otherwise control access of the data plane VCN 1118 to any outside networks or databases. Applying filters and controls by the customer onto the data plane VCN 1118, contained in the customer tenancy 1121, can help isolate the data plane VCN 1118 from other customers and from public Internet 1154.

In some embodiments, cloud services 1156 can be called by the service gateway 1136 to access services that may not exist on public Internet 1154, on the control plane VCN 1116, or on the data plane VCN 1118. The connection between cloud services 1156 and the control plane VCN 1116 or the data plane VCN 1118 may not be live or continuous. Cloud services 1156 may exist on a different network owned or operated by the IaaS provider. Cloud services 1156 may be configured to receive calls from the service gateway 1136 and may be configured to not receive calls from public Internet 1154. Some cloud services 1156 may be isolated from other cloud services 1156, and the control plane VCN 1116 may be isolated from cloud services 1156 that may not be in the same region as the control plane VCN 1116. For example, the control plane VCN 1116 may be located in “Region 1,” and cloud service “Deployment 10,” may be located in Region 1 and in “Region 2.” If a call to Deployment 10 is made by the service gateway 1136 contained in the control plane VCN 1116 located in Region 1, the call may be transmitted to Deployment 10 in Region 1. In this example, the control plane VCN 1116, or Deployment 10 in Region 1, may not be communicatively coupled to, or otherwise in communication with, Deployment 10 in Region 2.

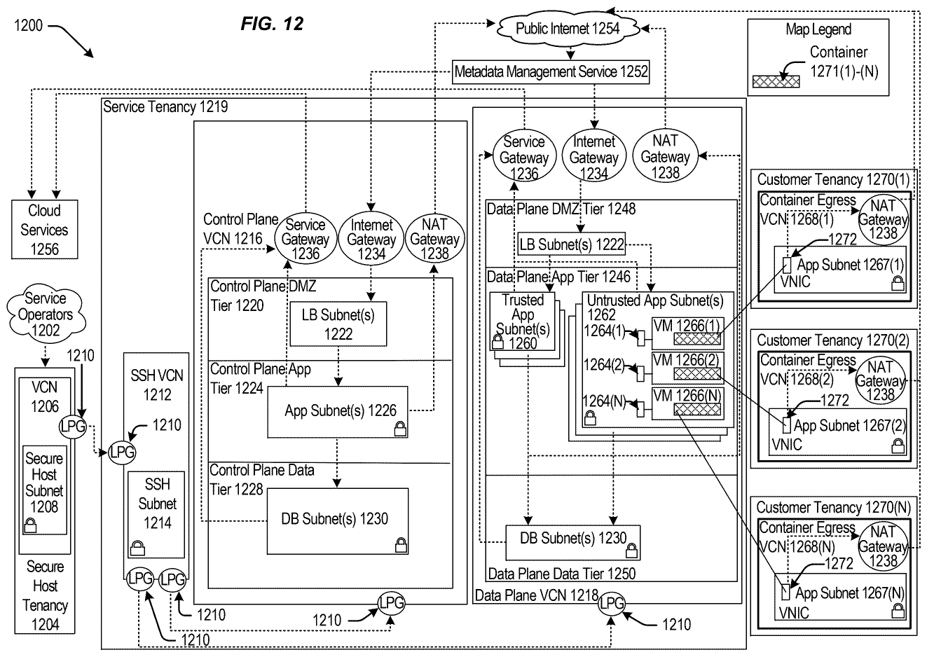

FIG. 12 is a block diagram 1200 illustrating another example pattern of an IaaS architecture, according to at least one embodiment. Service operators 1202 (e.g., service operators 1002 of FIG. 10) can be communicatively coupled to a secure host tenancy 1204 (e.g., the secure host tenancy 1004 of FIG. 10) that can include a virtual cloud network (VCN) 1206 (e.g., the VCN 1006 of FIG. 10) and a secure host subnet 1208 (e.g., the secure host subnet 1008 of FIG. 10). The VCN 1206 can include an LPG 1210 (e.g., the LPG 1010 of FIG. 10) that can be communicatively coupled to an SSH VCN 1212 (e.g., the SSH VCN 1012 of FIG. 10) via an LPG 1210 contained in the SSH VCN 1212. The SSH VCN 1212 can include an SSH subnet 1214 (e.g., the SSH subnet 1014 of FIG. 10), and the SSH VCN 1212 can be communicatively coupled to a control plane VCN 1216 (e.g., the control plane VCN 1016 of FIG. 10) via an LPG 1210 contained in the control plane VCN 1216 and to a data plane VCN 1218 (e.g., the data plane 1018 of FIG. 10) via an LPG 1210 contained in the data plane VCN 1218. The control plane VCN 1216 and the data plane VCN 1218 can be contained in a service tenancy 1219 (e.g., the service tenancy 1019 of FIG. 10).

The control plane VCN 1216 can include a control plane DMZ tier 1220 (e.g., the control plane DMZ tier 1020 of FIG. 10) that can include load balancer (LB) subnet(s) 1222 (e.g., LB subnet(s) 1022 of FIG. 10), a control plane app tier 1224 (e.g., the control plane app tier 1024 of FIG. 10) that can include app subnet(s) 1226 (e.g., similar to app subnet(s) 1026 of FIG. 10), a control plane data tier 1228 (e.g., the control plane data tier 1028 of FIG. 10) that can include DB subnet(s) 1230. The LB subnet(s) 1222 contained in the control plane DMZ tier 1220 can be communicatively coupled to the app subnet(s) 1226 contained in the control plane app tier 1224 and to an Internet gateway 1234 (e.g., the Internet gateway 1034 of FIG. 10) that can be contained in the control plane VCN 1216, and the app subnet(s) 1226 can be communicatively coupled to the DB subnet(s) 1230 contained in the control plane data tier 1228 and to a service gateway 1236 (e.g., the service gateway of FIG. 10) and a network address translation (NAT) gateway 1238 (e.g., the NAT gateway 1038 of FIG. 10). The control plane VCN 1216 can include the service gateway 1236 and the NAT gateway 1238.

The data plane VCN 1218 can include a data plane app tier 1246 (e.g., the data plane app tier 1046 of FIG. 10), a data plane DMZ tier 1248 (e.g., the data plane DMZ tier 1048 of FIG. 10), and a data plane data tier 1250 (e.g., the data plane data tier 1050 of FIG. 10). The data plane DMZ tier 1248 can include LB subnet(s) 1222 that can be communicatively coupled to trusted app subnet(s) 1260 and untrusted app subnet(s) 1262 of the data plane app tier 1246 and the Internet gateway 1234 contained in the data plane VCN 1218. The trusted app subnet(s) 1260 can be communicatively coupled to the service gateway 1236 contained in the data plane VCN 1218, the NAT gateway 1238 contained in the data plane VCN 1218, and DB subnet(s) 1230 contained in the data plane data tier 1250. The untrusted app subnet(s) 1262 can be communicatively coupled to the service gateway 1236 contained in the data plane VCN 1218 and DB subnet(s) 1230 contained in the data plane data tier 1250. The data plane data tier 1250 can include DB subnet(s) 1230 that can be communicatively coupled to the service gateway 1236 contained in the data plane VCN 1218.

The untrusted app subnet(s) 1262 can include one or more primary VNICs 1264(1)-(N) that can be communicatively coupled to tenant virtual machines (VMs) 1266(1)-(N). Each tenant VM 1266(1)-(N) can be communicatively coupled to a respective app subnet 1267(1)-(N) that can be contained in respective container egress VCNs 1268(1)-(N) that can be contained in respective customer tenancies 1270(1)-(N). Respective secondary VNICs 1272(1)-(N) can facilitate communication between the untrusted app subnet(s) 1262 contained in the data plane VCN 1218 and the app subnet contained in the container egress VCNs 1268(1)-(N). Each container egress VCNs 1268(1)-(N) can include a NAT gateway 1238 that can be communicatively coupled to public Internet 1254 (e.g., public Internet 1054 of FIG. 10).

The Internet gateway 1234 contained in the control plane VCN 1216 and contained in the data plane VCN 1218 can be communicatively coupled to a metadata management service 1252 (e.g., the metadata management system 1052 of FIG. 10) that can be communicatively coupled to public Internet 1254. Public Internet 1254 can be communicatively coupled to the NAT gateway 1238 contained in the control plane VCN 1216 and contained in the data plane VCN 1218. The service gateway 1236 contained in the control plane VCN 1216 and contained in the data plane VCN 1218 can be communicatively coupled to cloud services 1256.

In some embodiments, the data plane VCN 1218 can be integrated with customer tenancies 1270. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane app tier 1246. Code to run the function may be executed in the VMs 1266(1)-(N), and the code may not be configured to run anywhere else on the data plane VCN 1218. Each VM 1266(1)-(N) may be connected to one customer tenancy 1270. Respective containers 1271(1)-(N) contained in the VMs 1266(1)-(N) may be configured to run the code. In this case, there can be a dual isolation (e.g., the containers 1271(1)-(N) running code, where the containers 1271(1)-(N) may be contained in at least the VM 1266(1)-(N) that are contained in the untrusted app subnet(s) 1262), which may help prevent incorrect or otherwise undesirable code from damaging the network of the IaaS provider or from damaging a network of a different customer. The containers 1271(1)-(N) may be communicatively coupled to the customer tenancy 1270 and may be configured to transmit or receive data from the customer tenancy 1270. The containers 1271(1)-(N) may not be configured to transmit or receive data from any other entity in the data plane VCN 1218. Upon completion of running the code, the IaaS provider may kill or otherwise dispose of the containers 1271(1)-(N).

In some embodiments, the trusted app subnet(s) 1260 may run code that may be owned or operated by the IaaS provider. In this embodiment, the trusted app subnet(s) 1260 may be communicatively coupled to the DB subnet(s) 1230 and be configured to execute CRUD operations in the DB subnet(s) 1230. The untrusted app subnet(s) 1262 may be communicatively coupled to the DB subnet(s) 1230, but in this embodiment, the untrusted app subnet(s) may be configured to execute read operations in the DB subnet(s) 1230. The containers 1271(1)-(N) that can be contained in the VM 1266(1)-(N) of each customer and that may run code from the customer may not be communicatively coupled with the DB subnet(s) 1230.

In other embodiments, the control plane VCN 1216 and the data plane VCN 1218 may not be directly communicatively coupled. In this embodiment, there may be no direct communication between the control plane VCN 1216 and the data plane VCN 1218. However, communication can occur indirectly through at least one method. An LPG 1210 may be established by the IaaS provider that can facilitate communication between the control plane VCN 1216 and the data plane VCN 1218. In another example, the control plane VCN 1216 or the data plane VCN 1218 can make a call to cloud services 1256 via the service gateway 1236. For example, a call to cloud services 1256 from the control plane VCN 1216 can include a request for a service that can communicate with the data plane VCN 1218.

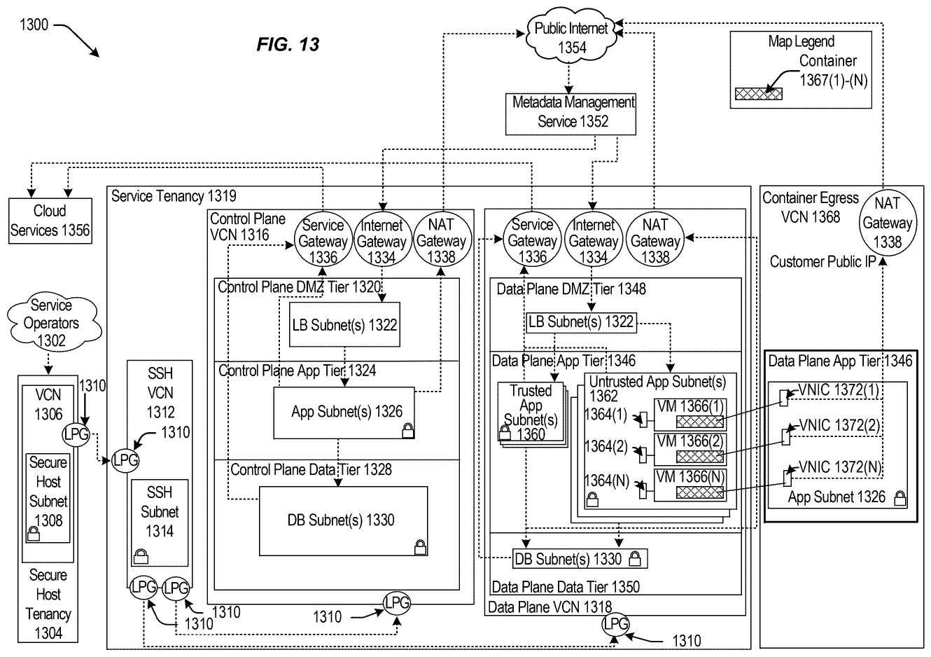

FIG. 13 is a block diagram 1300 illustrating another example pattern of an IaaS architecture, according to at least one embodiment. Service operators 1302 (e.g., service operators 1002 of FIG. 10) can be communicatively coupled to a secure host tenancy 1304 (e.g., the secure host tenancy 1004 of FIG. 10) that can include a virtual cloud network (VCN) 1306 (e.g., the VCN 1006 of FIG. 10) and a secure host subnet 1308 (e.g., the secure host subnet 1008 of FIG. 10). The VCN 1306 can include an LPG 1310 (e.g., the LPG 1010 of FIG. 10) that can be communicatively coupled to an SSH VCN 1312 (e.g., the SSH VCN 1012 of FIG. 10) via an LPG 1310 contained in the SSH VCN 1312. The SSH VCN 1312 can include an SSH subnet 1314 (e.g., the SSH subnet 1014 of FIG. 10), and the SSH VCN 1312 can be communicatively coupled to a control plane VCN 1316 (e.g., the control plane VCN 1016 of FIG. 10) via an LPG 1310 contained in the control plane VCN 1316 and to a data plane VCN 1318 (e.g., the data plane 1018 of FIG. 10) via an LPG 1310 contained in the data plane VCN 1318. The control plane VCN 1316 and the data plane VCN 1318 can be contained in a service tenancy 1319 (e.g., the service tenancy 1019 of FIG. 10).

The control plane VCN 1316 can include a control plane DMZ tier 1320 (e.g., the control plane DMZ tier 1020 of FIG. 10) that can include LB subnet(s) 1322 (e.g., LB subnet(s) 1022 of FIG. 10), a control plane app tier 1324 (e.g., the control plane app tier 1024 of FIG. 10) that can include app subnet(s) 1326 (e.g., app subnet(s) 1026 of FIG. 10), a control plane data tier 1328 (e.g., the control plane data tier 1028 of FIG. 10) that can include DB subnet(s) 1330 (e.g., DB subnet(s) 1230 of FIG. 12). The LB subnet(s) 1322 contained in the control plane DMZ tier 1320 can be communicatively coupled to the app subnet(s) 1326 contained in the control plane app tier 1324 and to an Internet gateway 1334 (e.g., the Internet gateway 1034 of FIG. 10) that can be contained in the control plane VCN 1316, and the app subnet(s) 1326 can be communicatively coupled to the DB subnet(s) 1330 contained in the control plane data tier 1328 and to a service gateway 1336 (e.g., the service gateway of FIG. 10) and a network address translation (NAT) gateway 1338 (e.g., the NAT gateway 1038 of FIG. 10). The control plane VCN 1316 can include the service gateway 1336 and the NAT gateway 1338.

The data plane VCN 1318 can include a data plane app tier 1346 (e.g., the data plane app tier 1046 of FIG. 10), a data plane DMZ tier 1348 (e.g., the data plane DMZ tier 1048 of FIG. 10), and a data plane data tier 1350 (e.g., the data plane data tier 1050 of FIG. 10). The data plane DMZ tier 1348 can include LB subnet(s) 1322 that can be communicatively coupled to trusted app subnet(s) 1360 (e.g., trusted app subnet(s) 1260 of FIG. 12) and untrusted app subnet(s) 1362 (e.g., untrusted app subnet(s) 1262 of FIG. 12) of the data plane app tier 1346 and the Internet gateway 1334 contained in the data plane VCN 1318. The trusted app subnet(s) 1360 can be communicatively coupled to the service gateway 1336 contained in the data plane VCN 1318, the NAT gateway 1338 contained in the data plane VCN 1318, and DB subnet(s) 1330 contained in the data plane data tier 1350. The untrusted app subnet(s) 1362 can be communicatively coupled to the service gateway 1336 contained in the data plane VCN 1318 and DB subnet(s) 1330 contained in the data plane data tier 1350. The data plane data tier 1350 can include DB subnet(s) 1330 that can be communicatively coupled to the service gateway 1336 contained in the data plane VCN 1318.

The untrusted app subnet(s) 1362 can include primary VNICs 1364(1)-(N) that can be communicatively coupled to tenant virtual machines (VMs) 1366(1)-(N) residing within the untrusted app subnet(s) 1362. Each tenant VM 1366(1)-(N) can run code in a respective container 1367(1)-(N), and be communicatively coupled to an app subnet 1326 that can be contained in a data plane app tier 1346 that can be contained in a container egress VCN 1368. Respective secondary VNICs 1372(1)-(N) can facilitate communication between the untrusted app subnet(s) 1362 contained in the data plane VCN 1318 and the app subnet contained in the container egress VCN 1368. The container egress VCN can include a NAT gateway 1338 that can be communicatively coupled to public Internet 1354 (e.g., public Internet 1054 of FIG. 10).

The Internet gateway 1334 contained in the control plane VCN 1316 and contained in the data plane VCN 1318 can be communicatively coupled to a metadata management service 1352 (e.g., the metadata management system 1052 of FIG. 10) that can be communicatively coupled to public Internet 1354. Public Internet 1354 can be communicatively coupled to the NAT gateway 1338 contained in the control plane VCN 1316 and contained in the data plane VCN 1318. The service gateway 1336 contained in the control plane VCN 1316 and contained in the data plane VCN 1318 can be communicatively coupled to cloud services 1356.

In some examples, the pattern illustrated by the architecture of block diagram 1300 of FIG. 13 may be considered an exception to the pattern illustrated by the architecture of block diagram 1200 of FIG. 12 and may be desirable for a customer of the IaaS provider if the IaaS provider cannot directly communicate with the customer (e.g., a disconnected region). The respective containers 1367(1)-(N) that are contained in the VMs 1366(1)-(N) for each customer can be accessed in real-time by the customer. The containers 1367(1)-(N) may be configured to make calls to respective secondary VNICs 1372(1)-(N) contained in app subnet(s) 1326 of the data plane app tier 1346 that can be contained in the container egress VCN 1368. The secondary VNICs 1372(1)-(N) can transmit the calls to the NAT gateway 1338 that may transmit the calls to public Internet 1354. In this example, the containers 1367(1)-(N) that can be accessed in real-time by the customer can be isolated from the control plane VCN 1316 and can be isolated from other entities contained in the data plane VCN 1318. The containers 1367(1)-(N) may also be isolated from resources from other customers.

In other examples, the customer can use the containers 1367(1)-(N) to call cloud services 1356. In this example, the customer may run code in the containers 1367(1)-(N) that requests a service from cloud services 1356. The containers 1367(1)-(N) can transmit this request to the secondary VNICs 1372(1)-(N) that can transmit the request to the NAT gateway that can transmit the request to public Internet 1354. Public Internet 1354 can transmit the request to LB subnet(s) 1322 contained in the control plane VCN 1316 via the Internet gateway 1334. In response to determining the request is valid, the LB subnet(s) can transmit the request to app subnet(s) 1326 that can transmit the request to cloud services 1356 via the service gateway 1336.

It should be appreciated that IaaS architectures 1000, 1100, 1200, 1300 depicted in the figures may have other components than those depicted. Further, the embodiments shown in the figures are only some examples of a cloud infrastructure system that may incorporate an embodiment of the disclosure. In some other embodiments, the IaaS systems may have more or fewer components than shown in the figures, may combine two or more components, or may have a different configuration or arrangement of components.

In certain embodiments, the IaaS systems described herein may include a suite of applications, middleware, and database service offerings that are delivered to a customer in a self-service, subscription-based, elastically scalable, reliable, highly available, and secure manner. An example of such an IaaS system is the Oracle Cloud Infrastructure (OCI) provided by the present assignee.

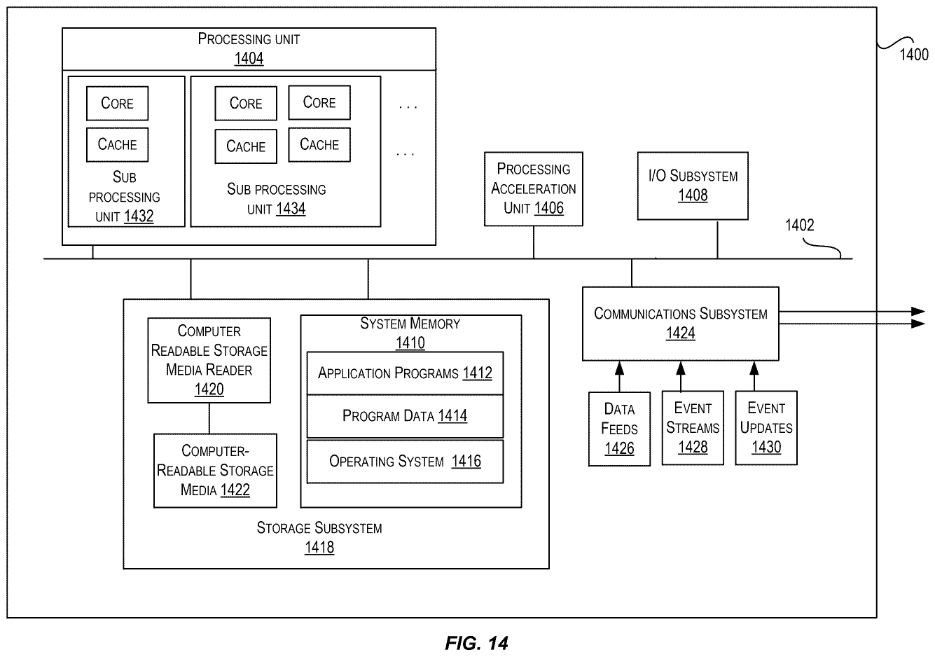

FIG. 14 illustrates an example computer system 1400, in which various embodiments may be implemented. The system 1400 may be used to implement any of the computer systems described above. As shown in the figure, computer system 1400 includes a processing unit 1404 that communicates with a number of peripheral subsystems via a bus subsystem 1402. These peripheral subsystems may include a processing acceleration unit 1406, an I/O subsystem 1408, a storage subsystem 1418 and a communications subsystem 1424. Storage subsystem 1418 includes tangible computer-readable storage media 1422 and a system memory 1410.

Bus subsystem 1402 provides a mechanism for letting the various components and subsystems of computer system 1400 communicate with each other as intended. Although bus subsystem 1402 is shown schematically as a single bus, alternative embodiments of the bus subsystem may utilize multiple buses. Bus subsystem 1402 may be any of several types of bus structures including a memory bus or memory controller, a peripheral bus, and a local bus using any of a variety of bus architectures. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard.

Processing unit 1404, which can be implemented as one or more integrated circuits (e.g., a conventional microprocessor or microcontroller), controls the operation of computer system 1400. One or more processors may be included in processing unit 1404. These processors may include single core or multicore processors. In certain embodiments, processing unit 1404 may be implemented as one or more independent processing units 1432 and/or 1434 with single or multicore processors included in each processing unit. In other embodiments, processing unit 1404 may also be implemented as a quad-core processing unit formed by integrating two dual-core processors into a single chip.

In various embodiments, processing unit 1404 can execute a variety of programs in response to program code and can maintain multiple concurrently executing programs or processes. At any given time, some or all of the program code to be executed can be resident in processor(s) 1404 and/or in storage subsystem 1418. Through suitable programming, processor(s) 1404 can provide various functionalities described above. Computer system 1400 may additionally include a processing acceleration unit 1406, which can include a digital signal processor (DSP), a special-purpose processor, and/or the like.

I/O subsystem 1408 may include user interface input devices and user interface output devices. User interface input devices may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may include, for example, motion sensing and/or gesture recognition devices such as the Microsoft Kinect® motion sensor that enables users to control and interact with an input device, such as the Microsoft Xbox® 360 game controller, through a natural user interface using gestures and spoken commands. User interface input devices may also include eye gesture recognition devices such as the Google Glass® blink detector that detects eye activity (e.g., ‘blinking’ while taking pictures and/or making a menu selection) from users and transforms the eye gestures as input into an input device (e.g., Google Glass®). Additionally, user interface input devices may include voice recognition sensing devices that enable users to interact with voice recognition systems (e.g., Siri® navigator), through voice commands.

User interface input devices may also include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices such as computed tomography, magnetic resonance imaging, position emission tomography, medical ultrasonography devices. User interface input devices may also include, for example, audio input devices such as MIDI keyboards, digital musical instruments and the like.

User interface output devices may include a display subsystem, indicator lights, or non-visual displays such as audio output devices, etc. The display subsystem may be a cathode ray tube (CRT), a flat-panel device, such as that using a liquid crystal display (LCD) or plasma display, a projection device, a touch screen, and the like. In general, use of the term “output device” is intended to include all possible types of devices and mechanisms for outputting information from computer system 1400 to a user or other computer. For example, user interface output devices may include, without limitation, a variety of display devices that visually convey text, graphics and audio/video information such as monitors, printers, speakers, headphones, automotive navigation systems, plotters, voice output devices, and modems.

Computer system 1400 may comprise a storage subsystem 1418 that provides a tangible non-transitory computer-readable storage medium for storing software and data constructs that provide the functionality of the embodiments described in this disclosure. The software can include programs, code modules, instructions, scripts, etc., that when executed by one or more cores or processors of processing unit 1404 provide the functionality described above. Storage subsystem 1418 may also provide a repository for storing data used in accordance with the present disclosure.

As depicted in the example in FIG. 14, storage subsystem 1418 can include various components including a system memory 1410, computer-readable storage media 1422, and a computer readable storage media reader 1420. System memory 1410 may store program instructions that are loadable and executable by processing unit 1404. System memory 1410 may also store data that is used during the execution of the instructions and/or data that is generated during the execution of the program instructions. Various different kinds of programs may be loaded into system memory 1410 including but not limited to client applications, Web browsers, mid-tier applications, relational database management systems (RDBMS), virtual machines, containers, etc.

System memory 1410 may also store an operating system 1416. Examples of operating system 1416 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® OS, and Palm® OS operating systems. In certain implementations where computer system 1400 executes one or more virtual machines, the virtual machines along with their guest operating systems (GOSs) may be loaded into system memory 1410 and executed by one or more processors or cores of processing unit 1404.

System memory 1410 can come in different configurations depending upon the type of computer system 1400. For example, system memory 1410 may be volatile memory (such as random access memory (RAM)) and/or non-volatile memory (such as read-only memory (ROM), flash memory, etc.) Different types of RAM configurations may be provided including a static random access memory (SRAM), a dynamic random access memory (DRAM), and others. In some implementations, system memory 1410 may include a basic input/output system (BIOS) containing basic routines that help to transfer information between elements within computer system 1400, such as during start-up.

Computer-readable storage media 1422 may represent remote, local, fixed, and/or removable storage devices plus storage media for temporarily and/or more permanently containing, storing, computer-readable information for use by computer system 1400 including instructions executable by processing unit 1404 of computer system 1400.

Computer-readable storage media 1422 can include any appropriate media known or used in the art, including storage media and communication media, such as but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media.

By way of example, computer-readable storage media 1422 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 1422 may include, but is not limited to, Zip® drives, flash memory cards, universal serial bus (USB) flash drives, secure digital (SD) cards, DVD disks, digital video tape, and the like. Computer-readable storage media 1422 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 1400.

Machine-readable instructions executable by one or more processors or cores of processing unit 1404 may be stored on a non-transitory computer-readable storage medium. A non-transitory computer-readable storage medium can include physically tangible memory or storage devices that include volatile memory storage devices and/or non-volatile storage devices. Examples of non-transitory computer-readable storage medium include magnetic storage media (e.g., disk or tapes), optical storage media (e.g., DVDs, CDs), various types of RAM, ROM, or flash memory, hard drives, floppy drives, detachable memory drives (e.g., USB drives), or other type of storage device.

Communications subsystem 1424 provides an interface to other computer systems and networks. Communications subsystem 1424 serves as an interface for receiving data from and transmitting data to other systems from computer system 1400. For example, communications subsystem 1424 may enable computer system 1400 to connect to one or more devices via the Internet. In some embodiments communications subsystem 1424 can include radio frequency (RF) transceiver components for accessing wireless voice and/or data networks (e.g., using cellular telephone technology, advanced data network technology, such as 3G, 4G or EDGE (enhanced data rates for global evolution), WiFi (IEEE 802.11 family standards, or other mobile communication technologies, or any combination thereof)), global positioning system (GPS) receiver components, and/or other components. In some embodiments communications subsystem 1424 can provide wired network connectivity (e.g., Ethernet) in addition to or instead of a wireless interface.

In some embodiments, communications subsystem 1424 may also receive input communication in the form of structured and/or unstructured data feeds 1426, event streams 1428, event updates 1430, and the like on behalf of one or more users who may use computer system 1400.

By way of example, communications subsystem 1424 may be configured to receive data feeds 1426 in real-time from users of social networks and/or other communication services such as Twitter® feeds, Facebook® updates, web feeds such as Rich Site Summary (RSS) feeds, and/or real-time updates from one or more third party information sources.

Additionally, communications subsystem 1424 may also be configured to receive data in the form of continuous data streams, which may include event streams 1428 of real-time events and/or event updates 1430, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1424 may also be configured to output the structured and/or unstructured data feeds 1426, event streams 1428, event updates 1430, and the like to one or more databases that may be in communication with one or more streaming data source computers coupled to computer system 1400.

Computer system 1400 can be one of various types, including a handheld portable device (e.g., an iPhone® cellular phone, an iPad® computing tablet, a PDA), a wearable device (e.g., a Google Glass® head mounted display), a PC, a workstation, a mainframe, a kiosk, a server rack, or any other data processing system.

Due to the ever-changing nature of computers and networks, the description of computer system 1400 depicted in the figure is intended only as a specific example. Many other configurations having more or fewer components than the system depicted in the figure are possible. For example, customized hardware might also be used and/or particular elements might be implemented in hardware, firmware, software (including applets), or a combination. Further, connection to other computing devices, such as network input/output devices, may be employed. Based on the disclosure and teachings provided herein, a person of ordinary skill in the art will appreciate other ways and/or methods to implement the various embodiments.

Although specific embodiments have been described, various modifications, alterations, alternative constructions, and equivalents are also encompassed within the scope of the disclosure. Embodiments are not restricted to operation within certain specific data processing environments, but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or services are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known for carrying out the disclosure. Variations of those preferred embodiments may become apparent to those of ordinary skill in the art upon reading the foregoing description. Those of ordinary skill should be able to employ such variations as appropriate and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein.

All references, including publications, patent applications, and patents, cited herein are hereby incorporated by reference to the same extent as if each reference were individually and specifically indicated to be incorporated by reference and were set forth in its entirety herein.

In the foregoing specification, aspects of the disclosure are described with reference to specific embodiments thereof, but those skilled in the art will recognize that the disclosure is not limited thereto. Various features and aspects of the above-described disclosure may be used individually or jointly. Further, embodiments can be utilized in any number of environments and applications beyond those described herein without departing from the broader spirit and scope of the specification. The specification and drawings are, accordingly, to be regarded as illustrative rather than restrictive.

What is claimed is:

1. A computer-implemented method for executing queries with semantic code expansion in a clinical data system, comprising:
receiving a query containing a predicate specifying a clinical code and a semantic expansion parameter indicating a request for approximate matching;
retrieving a vector embedding associated with the specified clinical code from a pre-computed embedding index;
performing, based on the vector embedding, a similarity search in a vector space to identify one or more semantically similar clinical codes;
retrieving one or more exact code mappings for the specified clinical code from a mapping registry;
generating a rewritten query predicate comprising (i) the one or more exact code mappings as primary match conditions and (ii) the one or more semantically similar clinical codes as secondary match conditions;
executing the rewritten query against a clinical data store to return results matching (i) the one or more exact code mappings, (ii) the one or more semantically similar clinical codes, or (iii) both; and
annotating the results to distinguish between exact matches and semantic matches.

2. The computer-implemented method of claim 1, wherein performing the similarity search comprises:
computing a set of similarity scores wherein each similarity score of the set of similarity scores is computed between the retrieved vector embedding and a vector embedding of a candidate code; and
selecting the one or more semantically similar clinical codes by identifying one or more similarity scores of the set of similarity scores that exceed a threshold.

3. The computer-implemented method of claim 1, wherein the mapping registry comprises a mapping table including hash-based indexes, and wherein retrieving the one or more exact code mappings comprises performing a point lookup based on a hash index of the clinical code.

4. The computer-implemented method of claim 1, wherein the mapping registry comprises a hierarchical graph stored in a graph database, and wherein retrieving the one or more exact code mappings comprises performing a traversal of the hierarchical graph based on the clinical code and one or more coding systems to identify the one or more exact code mappings.

5. The computer-implemented method of claim 1, wherein the query comprises one or more constructs indicating at least one of (i) a coding system associated with the clinical code or (ii) a target coding system for approximate matching.

6. The computer-implemented method of claim 1, wherein the semantic expansion parameter indicates at least one of (i) a type of semantic expansion or (ii) a number of candidate codes.

7. The computer-implemented method of claim 1, wherein:
the query indicates a specified version of the clinical code, wherein the specified version corresponds to a historical value of the clinical code;
retrieving the one or more exact code mappings comprises retrieving one or more exact code mappings associated with the specified version of the clinical code;
the retrieved vector embedding is associated with the specified version of the clinical code; and
the results are annotated with mapping version metadata.

8. The computer-implemented method of claim 1, further comprising:
determining, for each result matching the one or more semantically similar codes, a relevance score; and
annotating the results with the relevance score for each semantic match.

9. The computer-implemented method of claim 1, wherein the clinical data system comprises a plurality of clinical data stores, and wherein the computer-implemented method further comprises:
retrieving results matching the exact code mappings from a relational data store of the plurality of clinical data stores; and
retrieving results matching the semantically similar clinical codes from a vector data store of the plurality of clinical data stores.

10. A system comprising:
a clinical data system comprising a clinical data store;
one or more processors; and
one or more computer-readable media storing instructions which, when executed by the one or more processors, cause the system to perform operations comprising:
receiving a query containing a predicate specifying a clinical code and a semantic expansion parameter indicating a request for approximate matching;
retrieving a vector embedding associated with the specified clinical code from a pre-computed embedding index;
performing, based on the vector embedding, a similarity search in a vector space to identify one or more semantically similar clinical codes;
retrieving one or more exact code mappings for the specified clinical code from a mapping registry;
generating a rewritten query predicate comprising (i) the one or more exact code mappings as primary match conditions and (ii) the one or more semantically similar clinical codes as secondary match conditions;
executing the rewritten query against the clinical data store to return results matching (i) the one or more exact code mappings, (ii) the one or more semantically similar clinical codes, or (iii) both; and
annotating the results to distinguish between exact matches and semantic matches.

11. The system of claim 10, wherein performing the similarity search comprises:
computing a set of similarity scores wherein each similarity score of the set of similarity scores is computed between the retrieved vector embedding and a vector embedding of a candidate code; and
selecting the one or more semantically similar clinical codes by identifying one or more similarity scores of the set of similarity scores that exceed a threshold.

12. The system of claim 10, wherein the mapping registry comprises a hierarchical graph stored in a graph database, and wherein retrieving the one or more exact code mappings comprises performing a traversal of the hierarchical graph based on the clinical code and one or more coding systems to identify the one or more exact code mappings.

13. The system of claim 10, wherein the query comprises one or more constructs indicating at least one of (i) a coding system associated with the clinical code or (ii) a target coding system for approximate matching.

14. The system of claim 10, wherein:
the query indicates a specified version of the clinical code, wherein the specified version corresponds to a historical value of the clinical code;
retrieving the one or more exact code mappings comprises retrieving one or more exact code mappings associated with the specified version of the clinical code;
the retrieved vector embedding is associated with the specified version of the clinical code; and
the results are annotated with mapping version metadata.

15. The system of claim 10, wherein the operations further comprise:
determining, for each result matching the one or more semantically similar codes, a relevance score; and
annotating the results with the relevance score for each semantic match.

16. One or more non-transitory computer-readable media storing instructions which, when executed by one or more processors, cause the one or more processors to perform operations comprising:
receiving a query containing a predicate specifying a clinical code and a semantic expansion parameter indicating a request for approximate matching;
retrieving a vector embedding associated with the specified clinical code from a pre-computed embedding index;
performing, based on the vector embedding, a similarity search in a vector space to identify one or more semantically similar clinical codes;
retrieving one or more exact code mappings for the specified clinical code from a versioned mapping registry;
generating a rewritten query predicate comprising (i) the one or more exact code mappings as primary match conditions and (ii) the one or more semantically similar clinical codes as secondary match conditions;
executing the rewritten query against a clinical data store to return results matching (i) the one or more exact code mappings, (ii) the one or more semantically similar clinical codes, or (iii) both; and
annotating the results to distinguish between exact matches and semantic matches.

17. The one or more non-transitory computer-readable media of claim 16, wherein performing the similarity search comprises:
computing a set of similarity scores wherein each similarity score of the set of similarity scores is computed between the retrieved vector embedding and a vector embedding of a candidate code; and
selecting the one or more semantically similar clinical codes by identifying one or more similarity scores of the set of similarity scores that exceed a threshold.

18. The one or more non-transitory computer-readable media of claim 16, wherein the query comprises one or more constructs indicating at least one of (i) a coding system associated with the clinical code or (ii) a target coding system for approximate matching.

19. The one or more non-transitory computer-readable media of claim 16, wherein:
the query indicates a specified version of the clinical code, wherein the specified version corresponds to a historical value of the clinical code;
retrieving the one or more exact code mappings comprises retrieving one or more exact code mappings associated with the specified version of the clinical code;
the retrieved vector embedding is associated with the specified version of the clinical code; and
the results are annotated with mapping version metadata.

20. The one or more non-transitory computer-readable media of claim 16, wherein the operations further comprise:
determining, for each result matching the one or more semantically similar codes, a relevance score; and
annotating the results with the relevance score for each semantic match.
